(19) United States
(12) Patent Application Publication
(10) Pub. No.: US 2024/0307105 A1
(43) Pub. Date: Sep. 19, 2024

(54) SYSTEMS AND METHODS FOR TREATING JOINTS

(71) Applicant: Artezo, Inc., Carmel, CA (US)

(72) Inventors: Travis Jered Nolan, Carmel, CA (US); Keir David Hart, Lafayette, CO (US); Russell Delmar Hempstead, Lafayette, CO (US)

(21) Appl. No.: 18/576,081

(22) PCT Filed: Jul. 6, 2022
(86) PCT No.: PCT/US2022/073450

§ 371 Date: Jan. 2, 2024

Related U.S. Application Data
(60) Provisional application No. 63/218,676, filed on Jul. 6, 2021.

(51) Int. Cl. A61B 18/14; A61B 18/00; A61B 18/12; A61B 90/00

(52) U.S. Cl. CPC A61B 18/1477; A61B 90/08; A61B 2018/00196; A61B 2018/00309; A61B 2018/00434; A61B 2018/00565; A61B 2018/00577; A61B 2018/00982; A61B 2018/126; A61B 2018/1425; A61B 2090/0811

(57) ABSTRACT

Methods and systems for modulating articular branch nerves emanating from sensory and/or motor nerves to treat joint pain. The methods and systems described may be used to modulate (e.g., denervate, ablate) articular branch nerves of, for example, cranial gluteal, femoral, sciatic, and/or obturator nerves, the sensory and/or motor nerves themselves, and/or other nerves innervating a joint. The modulation of these nerves may facilitate treatment of chronic joint pain (e.g., hip) and lameness (e.g., osteoarthritis (OA), degenerative joint disease (DJD), etc.). The modulation may be performed using a neuromodulation device (e.g., an energy delivery device). The device may use bipolar radiofrequency energy to form a linear or arcuate lesion. The lesion may follow curvature of bone, for example in a joint capsule.

### BACKGROUND

### Field

The present application is directed generally to medical devices, systems, and methods for treating tissue. More specifically, the application is directed to devices, systems, and methods for treating nerves, nerve fibers, or neurons to treat pain, and more particularly to therapeutically treat articular joint and/or synovial joint pain.

### Description of the Related Art

Osteoarthritis (OA) and degenerative joint disease (DJD) can affect both large and small animals. Non-human animals can begin developing the disease at a young age. The majority of OA in animals occur secondarily to developmental orthopedic disease, such as cranial cruciate ligament disease, hip dysplasia, elbow dysplasia, osteochondritis dissecans (OCD), patella (kneecap) dislocation, etc. In some animals, OA occurs with no obvious primary causes and can be related to genetics and age. Other contributing factors to OA can include, for example, body weight, obesity, gender, exercise, and diet.

Osteoarthritis generally begins as a disruption of the cartilage. Ultimately, this disruption causes the bones in the joint to erode into each other. Some signs or symptoms of osteoarthritis can include stiffness, lameness, and pain. The condition may start with minor pain during activity, but can develop into continuous chronic pain, which can even occur when the animal is at rest.

### SUMMARY

Joint pain can have deleterious effects such as musculoskeletal deterioration, central sensitization, and/or cognitive and/or affective decline, which can result in increased resistance to treatment. Using traditional treatment, once osteoarthritis has started in a joint, it generally cannot be fully cured and will likely affect an animal for the rest of his or her life. It is desirable to have new treatments for treating joints, in particular for treating joint pain. Preferably, such treatments would be minimally invasive, would reduce or negate the need for pharmaceutical remedies, and/or would be permanent or at least long-lasting.

Modulation (e.g., denervation, ablation, etc.) of one or multiple articular nerves that branch from sensory and/or motor nerves innervating the joint, for example by radiofrequency ablation and/or other modalities, can reduce or eliminate joint pain. For example, the articular branches of the sciatic nerve, the articular branches of the cranial gluteal nerve, the articular branches of the femoral nerve, and/or the articular branches of the obturator nerve maybe ablated using a minimally invasive percutaneous treatment device. The treatment device may be configured to deliver bipolar energy between two points of the treatment device. The modulation may be performed at a location along the articular nerve, for example at or near the end of the articular nerve located in a joint capsule, at a junction between the articular branch nerve and the nerve from which it emanates, and/or locations therebetween. Without being bound by any particular theory, the modulation of the articular nerve is akin to cutting a wire so that the pain transmission signal cannot be transmitted along the cut wire (the modulated articular nerve). The modulation procedure may be minimally invasive, for example using a needle or a device advanced through a needle or catheter.

In some embodiments, a minimally invasive method of modulating an articular nerve branch to treat hip joint pain in a subject comprises imaging the hip joint using at least one of ultrasound or fluoroscopy to identify anatomical landmarks, percutaneously inserting a treatment device to a first point determined by the anatomical landmarks until a distal end of the treatment device contacts bone, and extending a stylet out of a needle body of the treatment device. Extending the stylet comprises sliding the stylet along the bone to a second point. The needle body comprises a first electrode, and the stylet comprises a second electrode. The method comprises imaging using at least one of ultrasound or fluoroscopy to confirm a first location of the first electrode and the second electrode relative to the anatomical landmarks, and applying bipolar radiofrequency energy to the first electrode and the second electrode to ablate first tissue between the first point and the second point. The first tissue includes an articular branch nerve of a sciatic nerve. The method comprises retracting the stylet into the needle body, rotating the treatment device at the first point, and extending the stylet out of the needle body of the treatment device. Extending the stylet comprises sliding the stylet along the bone to a third point. The method comprises imaging using at least one of ultrasound or fluoroscopy to confirm a second location of the first electrode and the second electrode relative to the anatomical landmarks, and applying bipolar radiofrequency energy to the first electrode and the second electrode to ablate second tissue between the first point and the third point. The second tissue includes an articular branch nerve of a cranial gluteal nerve. The method comprises retracting the stylet into the needle body, and retracting the treatment device out of the subject.

In some embodiments, a minimally invasive method of modulating an articular nerve branch to treat hip joint pain in a subject comprises imaging the hip joint using at least one of ultrasound or fluoroscopy to identify anatomical landmarks, percutaneously inserting a treatment device to a first point determined by the anatomical landmarks until a distal end of the treatment device contacts bone, and extending a stylet out of a needle body of the treatment device. Extending the stylet comprises sliding the stylet along the bone to a second point. The needle body comprises a first electrode and the stylet comprising a second electrode. The method comprises imaging using at least one of ultrasound or fluoroscopy to confirm a first location of the first electrode and the second electrode relative to the anatomical landmarks, and applying bipolar radiofrequency energy to the first electrode and the second electrode to ablate first tissue between the first point and the second point. The first tissue includes an articular branch nerve of a sciatic nerve. The method comprises retracting the stylet into the needle body, retracting the treatment device out of the subject, percutaneously inserting the treatment device to a third point determined by the anatomical landmarks until the distal end of the treatment device contacts the bone, and extending the stylet out of the needle body of the treatment device. Extending the stylet comprises sliding the stylet along the bone to a fourth point. The method comprises imaging using at least one of ultrasound or fluoroscopy to confirm a second location of the first electrode and the second electrode relative to the anatomical landmarks, and applying bipolar radiofrequency energy to the first electrode and the second electrode to ablate second tissue between the third point and the fourth point. The second tissue includes an articular branch nerve of a cranial gluteal nerve. The method comprises retracting the stylet into the needle body, and retracting the treatment device out of the subject.

The above methods and other methods described herein may comprise percutaneously inserting the treatment device to a fifth point determined by the anatomical landmarks until a distal end of the treatment device contacts bone, and extending the stylet out of the needle body of the treatment device. Extending the stylet may comprise sliding the stylet along the bone to a sixth point. The method may comprise imaging using at least one of ultrasound or fluoroscopy to confirm a third location of the first electrode and the second electrode relative to the anatomical landmarks. The method may comprise applying bipolar radiofrequency energy to the first electrode and the second electrode to ablate third tissue between the fifth point and the sixth point. The third tissue may include an articular branch nerve of a femoral nerve. The method may comprise retracting the stylet into the needle body, and retracting the treatment device out of the subject.

The above methods or other methods described herein may comprise percutaneously inserting the treatment device to a seventh point determined by the anatomical landmarks until a distal end of the treatment device contacts bone, and extending the stylet out of the needle body of the treatment device. Extending the stylet may comprise sliding the stylet along the bone to an eighth point. The method may comprise imaging using at least one of ultrasound or fluoroscopy to confirm a fourth location of the first electrode and the second electrode relative to the anatomical landmarks. The method may comprise applying bipolar radiofrequency energy to the first electrode and the second electrode to ablate fourth tissue between the seventh point and the eighth point. The fourth tissue may include an articular branch nerve of an obturator nerve. The method may comprise retracting the stylet into the needle body, and retracting the treatment device out of the subject.

In some embodiments, a method of modulating an articular nerve branch to treat hip joint pain in a subject comprises percutaneously inserting a treatment device to a first point until a distal end of the treatment device contacts bone, and extending a stylet out of a needle body of the treatment device. Extending the stylet comprises sliding the stylet along the bone to a second point. The needle body comprises a first electrode and the stylet comprising a second electrode. The method comprises applying bipolar radiofrequency energy to the first electrode and the second electrode to ablate first tissue between the first point and the second point. The first tissue includes an articular branch nerve of a first one of a sciatic nerve, a cranial gluteal nerve, a femoral nerve, or an obturator nerve. The method comprises retracting the stylet into the needle body, and repeating the percutaneously inserting, extending, and applying to ablate second tissue including an articular branch nerve of a second one of the sciatic nerve, the cranial gluteal nerve, the femoral nerve, or the obturator nerve.

The first one may be the articular branch of the sciatic nerve and the second one may be the articular branch of the cranial gluteal nerve. The first one may be the articular branch of the cranial gluteal nerve and the second one may be the articular branch of the sciatic nerve. The method may comprise repeating the percutaneously inserting, extending, and applying to ablate second tissue including an articular branch nerve of a third one of the sciatic nerve, the cranial gluteal nerve, the femoral nerve, or the obturator nerve. The method may comprise repeating the percutaneously inserting, extending, and applying to ablate second tissue including an articular branch nerve of a fourth one of the sciatic nerve, the cranial gluteal nerve, the femoral nerve, or the obturator nerve.

In some embodiments, a minimally invasive method of modulating an articular nerve branch to treat pain in a joint of a subject comprises identifying an articular branch nerve emanating from a nerve innervating the joint (e.g., a sensory and/or motor nerve), and percutaneously modulating the articular branch nerve. After modulating the articular branch nerve, the pain is reduced.

The joint may comprise a hip. Identifying the articular branch nerve may comprise acquiring a hip joint reference point corresponding to a radiographically identifiable anatomical feature of the hip joint, and generating coordinates for a target treatment site as a function of a calculated distance from the reference point. The nerve may comprise at least one of the cranial gluteal nerve, femoral nerve, sciatic nerve, or obturator nerve. Modulating the articular branch nerve may comprise modulating the articular branch nerve at a target site. The target site may be external to the joint. The target site may comprise a portion of the articular branch nerve proximate a junction between the articular branch nerve and the nerve, a portion of the articular branch nerve proximate a terminus of the articular branch nerve, a portion of the articular branch nerve between a junction between the articular branch nerve and the nerve and a terminus of the articular branch nerve, and/or combinations thereof. Modulating the articular branch nerve may comprise percutaneously inserting a treatment device to a first point, extending a stylet out of a needle body of the treatment device, the needle body comprising a first electrode and the stylet comprising a second electrode, and applying energy to the treatment device to ablate tissue including the articular branch nerve. Applying the energy may comprise applying radiofrequency energy. The radiofrequency energy may be bipolar. The method may further comprise modulating the nerve.

In the above methods or other methods described herein, the subject may be a quadruped. The subject may be a canine. The subject may have osteoarthritis or degenerative joint disease.

In some embodiments, a device for treating tissue comprises, or alternatively consists essentially of, a needle body and a stylet configured to radially extend from the aperture of the needle body along a curved path, and a handle. The needle body comprises a first electrode, a tissue penetrating tip, a first temperature sensor, and an aperture in a side of the needle body. The stylet comprises a second electrode, a tissue penetrating tip, and a second temperature sensor. The handle is configured to be coupled to a tissue treatment system. The handle comprises a stylet mechanism configured to longitudinally move the stylet relative to the needle body.

The needle body may comprise a radiopaque material. The stylet may comprise a radiopaque material. The needle body may comprise an echogenic surface. The stylet may comprise an echogenic surface. The needle body may comprise stainless steel. The stylet may comprise a shape memory material. The shape memory material may comprise nitinol. The stylet mechanism may comprise a knob configured to slide along a path. The stylet mechanism may comprise the handle being rotatable relative to the needle body. The stylet mechanism may comprise a rotatable knob. The stylet mechanism may comprise indicia configured to inform a user about an extend of longitudinal movement of the stylet. The stylet mechanism may comprise detents configured to inform a user about an extend of longitudinal movement of the stylet. The device may comprise electronic circuitry configured to track use information. A kit may comprise the device and a guide catheter. The guide catheter may comprise a first lumen configured to accommodate passage of an imager, and a second lumen configured to accommodate passage of the device. A kit may comprise the device, and a tissue treatment system.

The tissue treatment system may comprise a display screen, an energy generator, a control computer, and a removable connector configured to couple the device and the system. The removable connector may comprise electronic circuitry configured to track use information. The tissue treatment system may comprise an imaging device.

In some embodiments, a device for treating tissue comprises, or alternatively consists essentially of, a needle body comprising a tissue penetrating tip and an aperture in a side of the needle body. The device comprises a first electrode and a stylet configured to radially extend from the aperture of the needle body. The stylet comprises a second electrode and a tissue penetrating tip. The device comprises a handle configured to be coupled to a tissue treatment system. The handle comprises a stylet mechanism configured to longitudinally move the stylet relative to the needle body.

The needle body may comprise a first temperature sensor. The stylet may comprise a second temperature sensor. The stylet may be configured to radially extend from the aperture of the needle body along a curved path. The stylet mechanism may comprise a knob configured to slide along a path. The stylet mechanism may comprise the handle being rotatable relative to the needle body. The stylet mechanism may comprise a rotatable knob. The needle body may comprise the first electrode. The tissue penetrating tip of the needle body may comprise the first electrode. The stylet may comprise the first electrode. The tissue penetrating tip of the stylet may comprise the first electrode. The stylet mechanism may comprise indicia configured to inform a user about an extend of longitudinal movement of the stylet. The stylet mechanism may comprise detents configured to inform a user about an extend of longitudinal movement of the stylet. The device may comprise electronic circuitry configured to track use information. The device may comprise a longitudinally movable ramp. The longitudinal position of the ramp may affect a longitudinal position at which the stylet radially extends from the aperture of the needle body. The handle may comprise a ramp mechanism configured to longitudinally move the ramp relative to the needle body. The stylet may be steerable. The stylet may be steerable in one direction. The stylet may be steerable in two directions. The two directions may be on one plane. The stylet may comprise one steering wire. The stylet may comprise two steering wires. The stylet may comprise a tube. The tube may comprise a first plurality of kerfs on a first side of the tube and a second plurality of kerfs on a second side of the tube opposite the first side. At least one of a shape, size, or spacing of the first plurality of kerfs may be different than at least one of a shape, size, or spacing of the second plurality of kerfs. A distal portion of the tube may comprise a pattern and material bent radially inwardly to attach a steering wire between the material and an inner surface of the tube. The stylet mechanism may comprise a knob configured to slide along a path and to rotate relative to the needle body. Rotation of the knob may be configured to steer the stylet. The stylet may comprise the first electrode and the second electrode longitudinally movable relative to each other. The stylet may comprise a first tube comprising the first electrode and a second tube comprising a second electrode. The second tube may be in telescoping arrangement with the first tube. The may comprise a tube comprising a shape memory material.

In some embodiments, a minimally invasive method of treating pain in an arcuate joint capsule of a subject comprises percutaneously positioning a treatment device at a first point along the capsule extending a stylet to a second point along the capsule, applying energy to the device to create a first lesion along the capsule, retracting the stylet, extending the stylet to a third point along the capsule, and applying energy to the device to create a second lesion along the capsule. The second lesion is at an angle to the first lesion.

The first lesion may have a thickness to length ratio between 1.25:1 and 10:1. The second lesion may have a thickness to length ratio between 1.25:1 and 10:1. The capsule may be a hip joint capsule. The subject may be a quadruped.

In some embodiments, a minimally invasive method of treating pain in a subject comprises positioning a treatment device at a first point and extending a stylet from the treatment device to a second point. After extending the stylet to the second point a first electrode is proximate to or touching a bone of the subject and the stylet comprises a second electrode proximate to or touching the bone. The method further comprises applying energy to the device to create a lesion along the bone. The lesion follows the curvature of the bone.

Applying the energy may comprise applying bipolar radiofrequency energy. The bone may include an arcuate rim. The treatment device may comprise the first electrode. The stylet may comprise the first electrode. The bone may be part of a hip joint capsule. The subject may be a quadruped.

In some embodiments, a treatment system comprises, consists essentially of, or consists of one or more of the features described herein.

In some embodiments, a tissue treatment system comprises, consists essentially of, or consists of one or more of the features described herein.

In some embodiments, a method of modulating an articulator nerve comprises, consists essentially of, or consists of one or more of the features described herein.

### BRIEF DESCRIPTION OF THE DRAWINGS

The following drawings are for illustrative purposes only and show non-limiting embodiments. Features from different figures may be combined in several embodiments.

FIG. 1A is a topographic illustration showing the innervation of the lateral canine hip joint.

FIG. 1B is a topographic illustration showing the innervation of the ventral canine hip joint.

FIG. 2 is a schematic drawing revealing articular branches of sciatic nerve supplying the dorsal portion of hip joint capsule.

FIG. 3 is an illustration displaying innervation of the craniolateral part of the hip joint capsule by the branches from the cranial gluteal nerve.

FIG. 4 is a diagram showing one of the articular branches of the femoral nerve innervating the cranioventral part of the hip joint capsule by passing through the m. iliopsoas.

FIG. 5 is a schematic drawing presenting the articular branch of the obturator nerve.

FIG. 6A schematically illustrates an example treatment device.

FIG. 6B schematically illustrates an example delivery device for a treatment device.

FIG. 7 is a schematic diagram of an example tissue treatment system.

FIGS. 8A and 8B illustrate an example method of treating hip joint pain.

FIG. 9A is a partial cross-sectional perspective view of an example tissue treatment device.

FIG. 9B is a partial perspective view of an example component of the tissue treatment device of FIG. 9A.

FIG. 9C is a partial cross-sectional side view of the tissue treatment device of FIG. 9A.

FIG. 9D is a schematic diagram of the tissue treatment device of FIG. 9A.

FIG. 9E is another schematic diagram of the tissue treatment device of FIG. 9A.

FIG. 10A is a partial perspective view of an example tissue treatment device.

FIG. 10B is a partial perspective view of the tissue treatment device of FIG. 10A with a component removed.

FIG. 10C is a partial cross-sectional distal end view of an example component of the tissue treatment device of FIG. 10A.

FIG. 10D is a partial perspective view of the tissue treatment device of FIG. 10A with additional components removed.

FIG. 10E is a partial cross-sectional side view of the tissue treatment device of FIG. 10A along the line 10E-10E.

FIG. 10F is a schematic diagram of the tissue treatment device of FIG. 10A.

FIG. 10G is another schematic diagram of the tissue treatment device of FIG. 10A.

FIG. 11A is a partial perspective view of an example tissue treatment device.

FIG. 11B is a partial perspective view of the tissue treatment device of FIG. 11A with a component removed.

FIG. 11Ci is a side view of an example component of the tissue treatment device of FIG. 11A.

FIG. 11Cii is an expanded side view of the example component of FIG. 11Ci.

FIG. 11D is a partial perspective view of the tissue treatment device of FIG. 11A with another component removed.

FIG. 11E is a partial cross-sectional side view of the tissue treatment device of FIG. 11A along the line 11E-11E.

FIG. 11Fi is a partial side and distal end perspective view of example components of the tissue treatment device of FIG. 11A.

FIG. 11Fii is a partial distal end view of the example components of FIG. 11Ei.

FIG. 12A is a partial perspective view of an example tissue treatment device.

FIG. 12B is a partial cross-sectional side view of the tissue treatment device of FIG. 12A along the line 12B-12B.

FIG. 12C is a schematic diagram of the tissue treatment device of FIG. 12A.

FIG. 12D is another schematic diagram of the tissue treatment device of FIG. 12A.

FIGS. 13A-13D are schematic depictions of example lesions.

FIG. 13E is another schematic depiction of an example lesion.

FIG. 14 is a schematic front (left) and side (right) depiction of example lesion.

FIG. 15A is an example method of treating hip joint pain.

FIG. 15B is another example method of treating hip joint pain.

### DETAILED DESCRIPTION

Joint pain that can be due to a variety of problems in both humans and non-human animals (e.g., canine, equine, feline, porcine, pachyderm, etc.) can be debilitating. Treatments for joint pain vary widely, and can include physical therapy, pharmacological therapy, surgical intervention, and/or others. Use of pharmacological therapy to treat joint pain can present several concerns. First, long term use can carry a high risk of complications to the animal's gastrointestinal tract, kidney, liver, and/or other organs. Second, the cost of the pharmacological therapy, usually over several years, may be extremely costly. Third, the pain generally persists for many years. Surgery can also present several concerns. First, most techniques disadvantageously involve removing part or most of the joint (osteotomy), surgically removing or transecting the nerves, and/or implanting a prosthesis. While surgery may provide long-term relief, surgical techniques have the disadvantage of being extremely expensive, having extensive recovery time, and being fraught with high complications rates (up to 20%). Additionally, many subjects (e.g., old, obese, etc.) may be unable to undergo surgery. While physical therapy does not necessarily present all the concerns of surgery or using pharmacological therapies, subjects receive varying degrees of pain relief, ranging from none or minimal to total. Additionally, physical therapy may provide only short-term pain relief, thereby extending treatment over several years, and thus increasing the cost of treatment. Moreover, many subjects ultimately require surgical intervention. Pharmaceuticals, surgery, and/or physical therapy may be combined with other treatments described herein, for example but not limited to use in a manner that may reduce or eliminate one, some, or all the concerns about such treatment.

Minimally invasive techniques for accessing the nerves have been developed through the surgical techniques used in regional nerve blocks, intra-articular joint injections, and articular denervation (e.g., ablation, neurectomy, etc.). Some procedures useful in humans do not translate well to other, non-human animal species (e.g., canine, equine, feline, porcine, pachyderm, etc.). Clear anatomical differences exist (e.g., different joints, nerves, access routes, sizes, etc.), but even how nerves innervate the joints is different. Different minimally invasive techniques of accessing the nerves are preferably used when treating non-human animals such as quadrupeds. In these species, minimally invasive techniques of accessing the nerves have been developed through surgical techniques useful in regional nerve blocks, intra-articular joint injections, and surgical joint capsule denervation (e.g., transection of articular nerve branches by surgically removing the periosteum).

The sensory and/or motor nerves have small nerve branches, known as articular nerves or articular nerve branches, that emanate from the trunk of the nerve and enter into the joint capsule or terminate in muscles adjacent to the joint capsule. Modulation of one articular nerve or a plurality of articular nerves can reduce or eliminate pain at that joint. The modulation may be performed at a location along the articular nerve at or near the end located in the joint capsule, for example to avoid modulating other anatomy, but modulation anywhere along the articular nerve may be effective. The modulation may be performed at a location along the articular nerve, for example at or near the end of the articular nerve located in a joint capsule, at a junction between the articular branch nerve and the nerve from which it emanates, and/or locations therebetween.

Hip joint arthritis (HJA) is one of the most common orthopedic problems in both humans and non-human animals (e.g., canine, equine, feline, porcine, pachyderm, etc.). Hip joint arthritis is usually accompanied by severe pain originating mainly from the richly innervated hip joint capsule. In canines, hip dysplasia is a common genetic disorder in which the ball and socket of the hip joint do not fit properly, causing rubbing and grinding instead of smooth sliding. A treatment based on modulation (e.g., inhibition, denervation, ablation, etc.) of articular nerves in and/or around the joint capsule may be a cost-effective alternative method for reduction of pain. For the subject or patient, this type of treatment can provide a significant improvement in the quality of life and, more importantly, significant slowing of the atrophy of pelvic limb muscles. Modulation (e.g., stimulation) of one or more nerves identified herein is provided in several embodiments. In some embodiments, reduction of neurotransmitter activity or release and/or increased neurotransmitter uptake or degradation is accomplished. In some embodiments, increased neurotransmitter activity or release and/or reduction of neurotransmitter uptake or degradation is accomplished. Examples of neurotransmitters include GABA, substance P, glutamate, dopamine, etc. In some embodiments, nociceptors are modulated. In several embodiments, the neuromodulation techniques described herein may be used synergistically with drugs or to replace drugs. This may advantageously reduce undesired side effects from drugs, dependence, tolerance, withdrawal, etc. In one embodiment, neuromodulation can reduce the dose, amount, time etc. of the drug required to achieve relief.

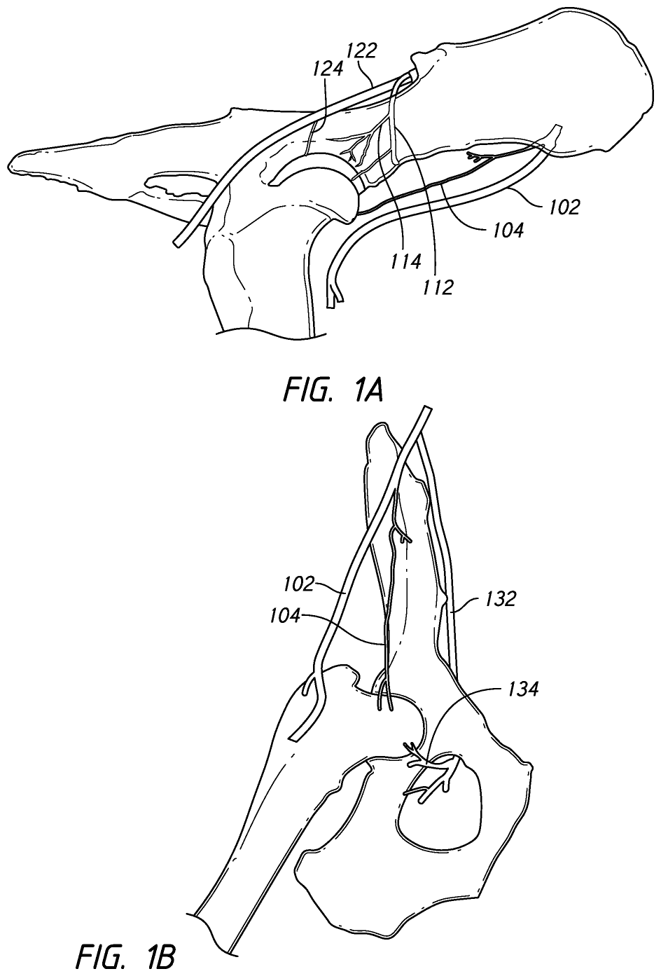

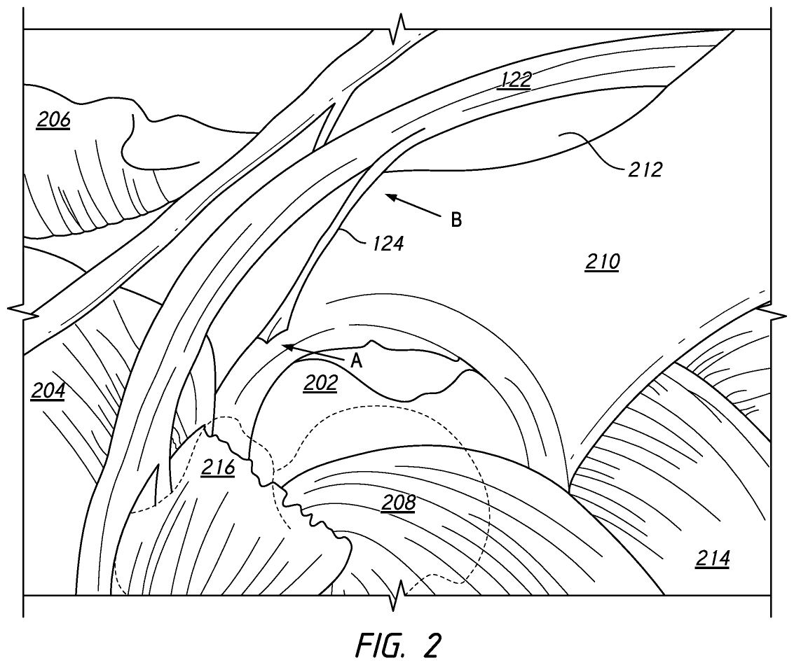

FIG. 1A is a topographic illustration showing the innervation of the lateral canine hip joint. FIG. 1B is a topographic illustration showing the innervation of the ventral canine hip joint. FIG. 1A shows a femoral nerve 102, articular branches 104 from femoral nerve 102, a cranial gluteal nerve 112, articular branches 114 from the cranial gluteal nerve 112, a sciatic nerve 122, and articular branches 124 from the sciatic nerve 122. FIG. 1B shows the femoral nerve 102, articular branches 104 from femoral nerve 102, an obturator nerve 132, and articular branches 134 from the obturator nerve 132. One, some, or all of the articular branches 104, 114, 124, 134 and/or one, some, or all of the nerves 102, 112, 122, 132 may be denervated to treat hip pain, several of which are described in additional detail herein. In some embodiments, the method does not include modulating a sensory nerve. In some embodiments, the method does not include modulating a motor nerve.

FIG. 2 is a schematic drawing revealing articular branches 124 of a sciatic nerve 122 supplying a dorsal portion of hip joint capsule 202 (right hip, lateral view). FIG. 2 shows the caudal part of m. gemelli 204, but the cranial part of mm. gemelli are not shown. The m. obturator internus 206 and the m. gluteus profundus 208 are shown elevated from the bone. For context, FIG. 2 also shows the body of ilium 210, the greater sciatic notch 212, the m. rectus femoris 214, and the greater trochanter 216. The terminus of the articular branch 124 is noted by the arrow A. The junction between the sciatic nerve 122 and the articular branch 124 is noted by the arrow B.

In some embodiments, a method of modulating an articular nerve branch 124 of a sciatic nerve 122 comprises locating a point on the sciatic nerve 122 as the sciatic nerve 122 passes through a caudal margin of a greater sciatic notch 212. The articular nerves 124 branch off after the point, and run to the dorsal portion of the hip joint capsule 202. The method further comprises modulating (e.g., denervating, ablating, etc.) the articular nerve branch 124.

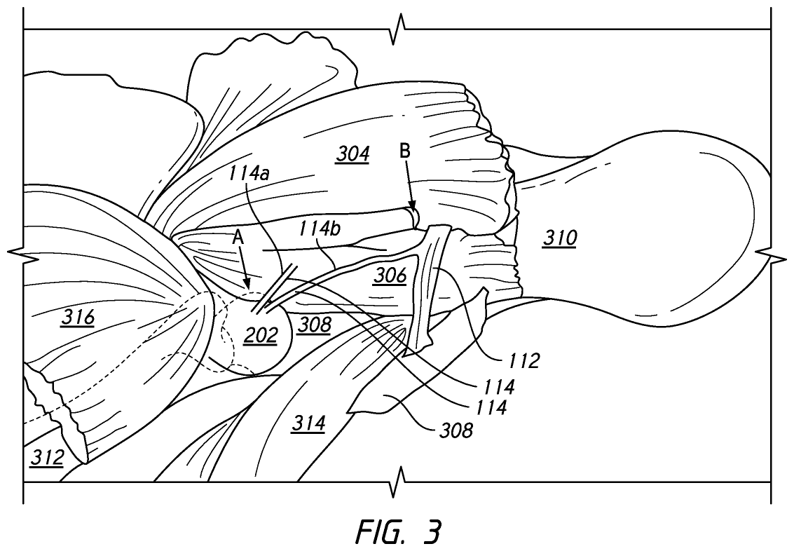

FIG. 3 is an illustration depicting innervation of a craniolateral section of a hip joint capsule 202 by articular nerve branches 114 emanating from a cranial gluteal nerve 112 (right hip, lateral view). The cranial gluteal nerve 112 penetrates between the m. gluteus medius 304 and m. gluteus profundus 306 and ends at the m. tensor fascia latae 308. The terminus of the articular branches 114 is noted by the arrow A. The junction between the gluteal nerve 112 and the articular branch 114 is noted by the arrow B. The junction noted by the arrow B may arise between the accessory head of the m. gluteus medius and the m. gluteus profundus. For context and clarity, FIG. 3 also illustrates the ilium 310, the femur 312, the m. rectus femoris 314, and the m. vastus lateralis 316, and the m. sartorius and m. articularis coxae are not shown. The cranial gluteal nerve 112 runs along the periosteum of the ilium 310 and enters the lateral muscles of the rump. The articular branch 114 may run vertically and caudally along the ventral fascia of the rump muscle before entering the hip joint capsule 202.

In several embodiments, a method of modulating an articular nerve branch 114 of a cranial gluteal nerve 112 comprises locating a junction B between the articular nerve branch 114 and a m. gluteus medius 304 and a m. gluteus profundus 306 after the articular nerve branch 114 enters lateral muscles of a rump. The articular nerve branch 114 may comprise a first articular branch 114a running between a periosteum and fascia of the m. gluteus profundus 306 running to a craniolateral aspect of the hip joint capsule 202. A second articular branch 114b can be identified before the cranial gluteal nerve 112 plunges into the m. tensor facia latae 308. The second articular branch 114 arises between the accessory head of the m. gluteus medius 304 and the m. gluteus profundus 306, and runs vertically and caudally along the ventral fascia of the rump muscle entering the craniolateral hip joint capsule and m. articularis coxae. The method further comprises modulating the articular nerve branches 114, for example as the articular nerve branches 114 enter a craniolateral aspect of the hip joint capsule 202.

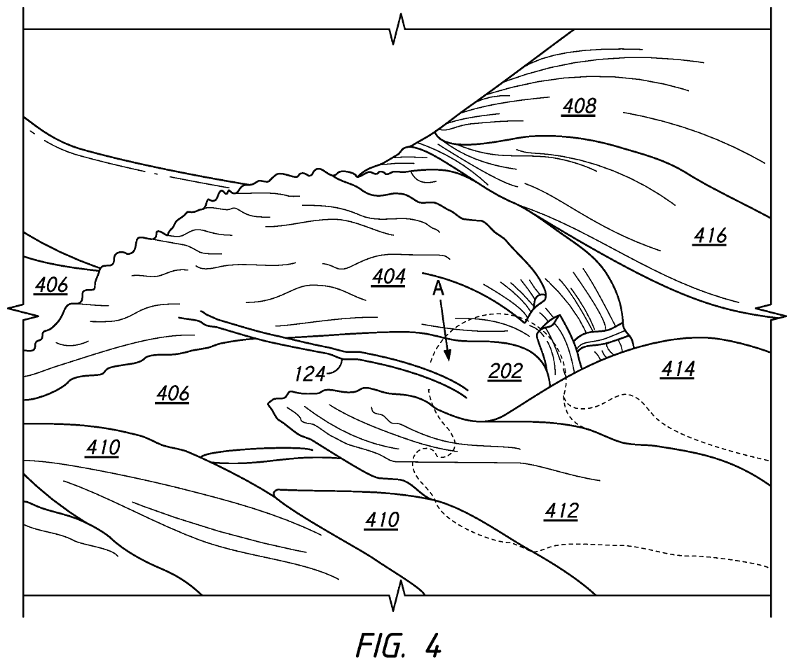

FIG. 4 is a diagram showing an articular branch 104 of a femoral nerve 102 (FIGS. 1A and 1B) innervating a cranioventral part of a hip joint capsule 202 by passing through the m. iliopsoas 404 (right hip with femur in full extension, ventral view). The terminus of the articular branch 104 is noted by the arrow A. For context and clarity, the m. iliopsoas 402 in FIG. 4 is partially severed from its original site, and FIG. 4 also shows an iliac body 406 (the m. iliopsoas 402 is elevated from the iliac body 406), mm. adductores 408, m. sartorius 410, m. rectus femoris 412, m. vastus medialis 414, and m. pectineus 416, and the femoral nerve and the femoral vessels are not shown.

In several embodiments, a method of modulating an articular nerve branch 104 of a femoral nerve 102 comprises locating a proximal portion of the femoral nerve 102 as the femoral nerve 102 passes caudoventrally through the m. iliopsoas 402. The articular branch 104 traverses distally to the caudal portion of the m. iliacus and then emerges out of the muscle, coursing a short distance between the fascia and the ventral periosteum of the ilium until reaching the cranioventral section of the hip joint capsule 402. The method further comprises modulating (e.g., denervating, ablating, etc.) the articular nerve branch 104.

FIG. 5 is a schematic drawing presenting an articular branch 134 of an obturator nerve 132 (right hip, caudoventral view with partial excision of pubis). The obturator nerve 132 passes through the obturator foramen. The terminus of the articular branch 134 is noted by the arrow A. The junction between the obturator nerve 132 and the articular branch 134 is noted by the arrow B. For context and clarity, FIG. 5 also shows a caudoventral portion of a hip joint capsule 202, muscular branches 504 of the obturator nerve 132 to mm. adductores, muscular branches 506 of the obturator nerve 132 to m. gracillus, m. obturator internus 508 separating from the pubis margin, and m. obturator externus 510 with partial resection, and branches of the medial circumflex femoral vessels are not shown.

In several embodiments, a method of modulating an articular nerve branch 134 of an obturator nerve 132 comprises following the obturator nerve 132 as the obturator nerve 132 passes through a cranial margin of obturator foramen, adjacent to a caudal portion of a hip joint capsule 202, into the obturator foramen. The articular nerve branch 134 emanates from the obturator nerve 132 when the obturator nerve 132 arrives into the foramen but before the obturator nerve 132 splits to form the muscular branches 504, 516 that feed into mm. adductores and mm. gracillus, respectively, and feeds into a caudal aspect of the hip joint capsule 202. The method further comprises modulating (e.g., denervating, ablating, etc.) the articular nerve branch 134.

In humans, the obturator nerve supplies the greatest part of the anterior capsule of the hip joint and is implicated as the cause of the groin and thigh portion of hip pain. Denervation of the obturator nerve plays an important role in managing of human hip pain. The Applicant has discovered that a connection between the obturator nerve and the source of hip pain in non-human animals, such as canine osteoarthritis, is not as strong such that denervation of the obturator nerve may not fully remedy hip pain or even play a significant role in addressing hip pain. In quadrupeds, access to the obturator nerve 132 may be difficult due to the anatomy. In some embodiments, the method does not include modulating the obturator nerve 132 or the articular branches 134 of the obturator nerve.

In several embodiments, methods are provided for treating joint pain associated with a hip of a subject. The articular joint (hip joint) is innervated by articular branch nerves originating or emanating from four nerves (cranial gluteal nerve, femoral nerve, sciatic nerve, and/or obturator nerve). In some embodiments, for example for treating quadrupeds, the method comprises identifying at least a portion of at least one of the four nerves that contribute to the circumambiency innervation of hip capsule and isolating the articular nerves at a location external to the joint capsule. In some embodiments, the method comprises modulating (e.g., denervating, ablating, etc.) the articular branch nerves at the external location with a neuromodulation device (e.g., energy delivery device) to treat pain associated with the hip.

In some embodiments, a method of modulating an articular nerve branch comprises identifying at least a portion of the articular nerve branch. Identifying the portion of the articular nerve branch comprises locating a nerve trunk and locating a junction between the articular nerve branch and the nerve trunk. The method further comprises modulating the articular nerve branch at or near the junction with the nerve trunk.

In some embodiments the method comprises identifying at least a portion of the articular nerves associated with a joint of the hip and isolating the articular nerve at a location external to the hip joint, comprising modulating (e.g., denervating, ablating, etc.) the articular nerve at the location external to the hip joint to treat the pain associated with the hip.

Several embodiments include a method for treating joint pain associated with a hip of a subject. The hip is innervated by articular branches originating from the cranial gluteal nerve, the sciatic nerve, the femoral nerve, and/or the obturator nerve. In some embodiments, the method comprises percutaneously guiding a delivery device within or near the hip joint. In some embodiments, the method comprises identifying at least a portion of the cranial gluteal nerve, femoral nerve, sciatic nerve, or obturator nerve associated with the hip joint and isolating the articular branch nerves at a location external to the hip joint. In some embodiments, the method comprises delivering a treatment device to the external location using the delivery device and operating the treatment device at the external location. In some embodiments, the operation of the treatment device is configured to modulate the cranial gluteal nerve, the femoral nerve, the sciatic nerve, or the obturator nerve at the external location to treat pain associated with the hip joint. In some embodiments, the delivery device comprises a catheter comprising a first lumen for advancing an imaging device and a second lumen for advancing the treatment device.

In some embodiments, determining a target treatment site comprises acquiring a hip joint reference point corresponding to a radiographically identifiable anatomical feature of the hip joint and generating coordinates for the target treatment site as a function of a calculated distance from the reference point. For example, the calculated distance may correspond to a predicted articular nerve location that is obtained (e.g., automatically obtained) from analysis of the acquired imaging data.

Treatment may be delivered to the target treatment site to modulate at least a portion of an articular nerve. The target treatment site may be, for example, a terminus of the articular nerve, a junction between the articular nerve and the nerve, and/or a portion of the articular nerve between the terminus and the junction. In accordance with several embodiments, the treatment may focus on a location of the articular nerve that is upstream (closer to the nerve junction, further from the terminus) of the articular nerve. In some embodiments, a method may comprise modulating (e.g., denervating, ablating, etc.) one or more nerves instead of or in addition to articular nerves. In some embodiments, a method does not include modulating a nerve.

In human beings, the sensitive innervation of the acetabular area includes branches from the superior gluteal, sciatic or ischiatic, femoral, and obturator nerves. In dogs, as an example of a quadruped, the sensitive innervation of the acetabular area includes the cranial gluteal, sciatic, and femoral nerves. The obturator nerve is not particularly related to this function in most non-human animals such that it can be considered an anatomic variation. The innervation, both in human beings and non-human animals, presents bilateral symmetry and, apart from that, the difference between the human bipedal support and the canine quadrupedal support generates biomechanical forces in distinct points, leading to different nerve fiber concentrations between the two species. While humans may present a bigger nerve fiber density in the anteromedial area, for which the obturator nerve is responsible, dogs present a bigger density in the craniolateral and dorsal areas, for which the cranial gluteal and the sciatic nerves are most responsible for pain, followed by the femoral nerve and the obturator nerve.

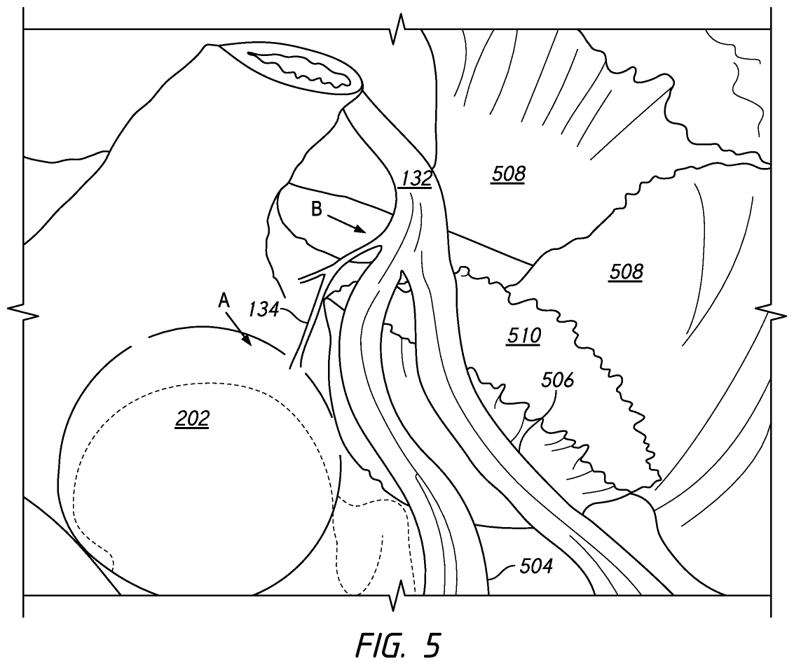

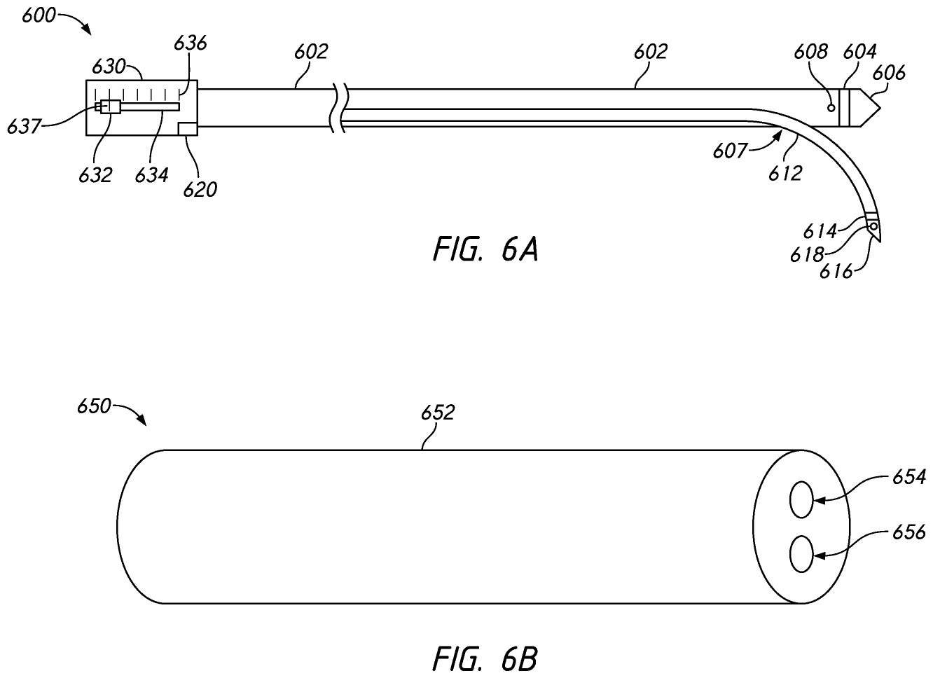

FIG. 6A schematically illustrates an example treatment device 600. The treatment device 600 comprises a needle body 602. The needle body 602 may comprise and suitable biocompatible material, for example, nitinol, chromium cobalt, Elgiloy, MP35N, Finox, Phynox, stainless steel, metal alloys, fiberglass, carbon fiber, etc. The first electrode 604 may comprise radiopaque material. The needle body 602 may comprise a radiopaque marker. For example, the tip 606 may comprise radiopaque material for visualization under fluoroscopy. The needle body 602 may comprise an echogenic surface. For example, the tip 606 may comprise an echogenic surface for visualization under ultrasound. In some embodiments, the tip 606 comprises radiopaque material and an echogenic surface. In contrast to direct visualization, for example as in open surgery, articular branches may be visualized which can provide targeted modulation of the desired nerves and not undesirably damage or remove a great deal of adjacent tissue.

The treatment device 600 comprises a first electrode 604 along the needle body 602. The first electrode 604 may at least partially span a circumference of the needle body 602. In some embodiments, the first electrode 604 comprises a barrel electrode. In some embodiments, the first electrode 604 comprises a button electrode. In some embodiments, the first electrode 604 comprises an uninsulated metal portion of the needle body 602. The first electrode 604 is proximate to a distal end of the needle body 602. For example, the first electrode 604 may be between about 0.1 mm and about 2 mm (e.g., about 0.1 mm, about 0.2 mm, about 0.3 mm, about 0.4 mm, about 0.5 mm, about 1 mm, about 1.5 mm, about 2.5 mm, and ranges between such values) from the distal end of the needle body 602. The distal end of the needle body 602 comprises a tissue-penetrating tip 606. The tip 606 is configured to allow the treatment device 600 to be inserted to a target site percutaneously or minimally invasively. In some embodiments, the procedures described herein, using the treatment device 600 or other treatment devices, can avoid open surgery and its myriad complications such as high infection risk, long recovery time of a 3-5 cm incision including all of the tissue between the skin and the bone, damaging or removing the entire periosteum, etc. The tip 606 may comprise, for example, a beveled tip (e.g., single bevel or multi-bevel), a pencil-point tip (e.g., multi-faceted or conical), etc. The treatment device 600 comprises a first temperature sensor 608 configured to sense a temperature proximate to the first electrode 604. The first temperature sensor 608 is shown proximal to the first electrode 604. Positioning the first temperature sensor 608 proximal to the first electrode 604 can reduce the distance between the first electrode 604 and the distal end of the tip 606 because there are fewer structures between the first electrode 604 and the distal end of the tip 606. Positioning the first temperature sensor 608 proximal to the first electrode 604 can be useful when power control is used for modulation because the temperature can be monitored closer to structures that are not trying to be treated (e.g., outside of an ablation zone). The first temperature sensor 608 may be distal to the first electrode 604. If the first electrode 604 is not fully annular, the first temperature sensor 608 may be longitudinally aligned with the first electrode 604.

The needle body 602 comprises an aperture 607. The needle body 602 may comprise a longitudinally-extending lumen in communication with the aperture 607. The needle body 602 may be hollow at least proximal to the aperture 607. The treatment device 600 comprises a stylet 612 configured to exit the aperture 607 and curve radially away from the needle body 602. The aperture 607 may be free of or lack a ramped surface. The stylet 612 may comprise and suitable biocompatible material, for example, nitinol, chromium cobalt, Elgiloy, MP35N, Finox, Phynox, stainless steel, metal alloys, etc. The stylet 612 preferably comprises a shape memory material so that the stylet 612 can be heat treated or shape set to have a curvature such that the stylet 612 radially extends from the aperture 607 along a curved path. The stylet 612 may comprise a shape memory material that, when the treatment device 600 is sterilized, reverts to a set curved shape, which may be able to reduce or eliminate bends in the stylet 612. The aperture 607 may be used to infuse liquid (e.g., saline, contrast, etc.) into a modulation site. For example, the modulation may comprise RF ablation enhanced with liquid.

The treatment device 600 comprises a second electrode 614 along the stylet 612. The second electrode 614 may at least partially span a circumference of the stylet 612. In some embodiments, the second electrode 614 comprises a barrel electrode. In some embodiments, the second electrode 614 comprises a button electrode. In some embodiments, the second electrode 614 comprises an uninsulated metal portion of the stylet 612. The second electrode 614 is proximate to a distal end of the stylet 612. The distal end of the stylet 612 comprises a tissue-penetrating tip 616. The tip 616 may comprise, for example, a beveled tip (e.g., single bevel or multi-bevel), a pencil-point tip (e.g., multi-faceted or conical), etc. In some embodiments, a beveled tip 616 that is angled to face the proximal end of the device 600 upon curving can deflect the stylet 612 distally as the stylet 612 is distally advanced. If the tip 606 contacts a bone (e.g., the ilium bone) and then the stylet 612 is distally advanced, a beveled tip 616 can help the distal part of the stylet 612 scrape along the surface of the bone. If the bevel faced distally, then the stylet 612 might be inclined to lift away from the bone. In some embodiments, a method of manufacturing the stylet 612 comprises forming a beveled tip 616 and shape setting a curve into the stylet 612 so that the beveled tip faces proximally upon curving. The curvature can help the stylet to extend substantially radially away from the needle body 602, for example with a small longitudinal component so that the first electrode 604 and the second electrode 614 can both be at a substantially similar longitudinal extent (e.g., along a bone).

The second electrode 614 may comprise radiopaque material. The stylet 612 may comprise a radiopaque marker. For example, the tip 606 may comprise radiopaque material for visualization under fluoroscopy. The stylet 612 may comprise an echogenic surface. For example, the tip 606 may comprise an echogenic surface for visualization under ultrasound. In some embodiments, the tip 606 comprises radiopaque material and an echogenic surface. Other radiopaque and/or echogenic markers are also possible. For example, the needle body 602 may comprise a radiopaque, echogenic, and/or visual marker to help a user identify the aperture 607 (e.g., proximate the aperture 607, around the aperture 607, on an opposite side of the aperture 607, etc.) to determine a direction of extension of the stylet 612. Fluoroscopy may be used to view radiopaque markers of the device 600 during and/or after insertion into the subject, for example to confirm position prior to modulation. Ultrasound may be used to view echogenic surfaces of the device 600 during and/or after insertion into the subject, for example to confirm position prior to modulation. The ultrasound may be external ultrasound. In some embodiments, the needle body 602 has a circular cross-section. In some embodiments, the needle body 602 has a non-circular cross-section, which can provide a user with rotational orientation information in the absence of a marker per se. For example, the needle body 602 can have a D-shaped cross-section in which the aperture 607 is along the flat side, opposite the flat side, etc.

The treatment device 600 comprises a second temperature sensor 618 configured to sense a temperature proximate to the second electrode 614. The second temperature sensor 618 is shown distal to the second electrode 614. Positioning the second temperature sensor 618 distal to the second electrode 614 does not affect the distance between the second electrode 614 and the distal lateral surface that the stylet 612 extends along. Positioning the second temperature sensor 618 distal to the second electrode 614 can be useful when power control is used for modulation because the temperature can be monitored closer to structures that are not trying to be treated (e.g., outside of an ablation zone). The second temperature sensor 618 may be proximal to the second electrode 614. If the second electrode 614 is not fully annular, the second temperature sensor 618 may be longitudinally aligned with the second electrode 614. The treatment device 600 may comprise the first temperature sensor 608, the second temperature sensor 618, or both the first temperature sensor 608 and the second temperature sensor 618. One temperature sensor may help to reduce costs and/or complexity of the treatment device 600. Two temperature sensors may help to provide information about specific local temperature (e.g., due to different heating proximate to the first electrode 604 versus the second electrode 614 due to nearby anatomy). Two temperature sensors may help to show an error in one of the temperature sensors. Other temperature sensors are also possible (e.g., proximate the aperture 607). The temperature sensor(s) may be used to provide feedback to a system that can be used, for example, to determine a treatment duration.

During modulation, the first electrode 604 may be an anode and the second electrode 614 may be a cathode, or vice versa, to provide bipolar modulation. The energy extends between the first electrode 604 and the second electrode 614 to create a line of ablation. In some embodiments, the treatment device 600 is configured to create a substantially cylindrical or thin prolate spheroid ablation zone. In some embodiments, the treatment device 600 does not create a spherical, egg-shaped, oval, etc. ablation zone. The modulation is preferably not monopolar and preferably does not use a grounding pad.

Other treatment devices are also possible. For example, the treatment device may comprise a needle and a plurality of stylets each comprising an electrode. Depending on positioning, one electrode on one stylet may be made anodic and one electrode on another stylet may be made cathodic so that the ablation zone is between the electrodes on two stylets (e.g., as opposite to being between an electrode on a needle body and an electrode on a stylet). If the stylets are configured to travel along the surface of a bone, for example, the ablation zone may be able to substantially avoid tissue distant from the bone. In some embodiments, a plurality of electrodes on different stylets may be used as the anode and/or the cathode, which can make generally flat polygonal shaped ablation zones, which could be useful for denervating a larger area where nerves extend in different directions. For another example, the treatment device may comprise a monopolar electrode. The monopolar electrode, for example when placed accurately relative to a nerve, can accurately ablate the nerve. A monopolar electrode may comprise a cooling system, for example if a larger ablation zone is desired. In some embodiments, the treatment device may be implantable. An implantable treatment device may advantageously modulate the nerves to inhibit or prevent transmission of pain signals, but keep the nerves intact. An implantable device may comprise a power source (e.g., wirelessly rechargeable battery) and a lead or a plurality of leads including electrodes configured to be positioned proximate the articular branch nerves of interest. In some embodiments, the modulation does not denervate or ablate tissue.

Although described herein with respect to RF (including bipolar, monopolar, liquid enhanced, etc.), other energy modalities may also or alternatively be used, for example unfocused ultrasound, focused ultrasound such as high-intensity or low-intensity focused ultrasound, microwave energy, thermal energy (e.g., cryoenergy, heat or cold provided by a fluid (e.g., water, saline, liquid medicament, etc.) or gas (e.g., steam)), electrical energy (e.g., non-RF electrical energy), infrared energy, laser energy, phototherapy or photodynamic therapy (e.g., in combination with one or more activation agents), plasma energy (e.g., plasma blades), ionizing energy delivery (e.g., X-ray, proton beam, gamma rays, electron beams, alpha rays, etc.), electroporation (e.g., irreversible electroporation), mechanical energies delivered by cutting or abrasive elements, cryoablation, chemical energy or modulation (e.g., chemoablation), or combinations thereof. In some embodiments, disruption or interruption of nerves is carried out by chemicals or therapeutic agents (for example, via drug delivery), either alone or in combination with an energy modality. In some embodiments, pharmaceuticals are combined with the neuromodulation (e.g., ablation) described herein to reduce the dosage or duration of pharmacology therapy, thus reducing side effects. In various embodiments, different energy modalities may be used in combination (either simultaneously or sequentially).

In some embodiments, the device 600 comprises a handle 630. The handle 630 is configured to couple to a nerve modulation system (e.g., by connecting the treatment device 600 to a connector). The handle 630 may comprise, for example, electrical couplers and/or fluid couplers. The handle 630 comprises a mechanism for deploying the stylet 612 from the needle body 602. FIG. 6A shows an example deployment mechanism comprising a knob 632 configured to slide in a channel 634. Distally advancing the knob 632 distally advances the stylet 612. The movement may be direct, or gearing may be used such that the movement of the knob 632 is larger than the movement of the stylet 612 or smaller than the movement of the stylet 612. The handle 630 may comprise indicia 636, for example alignable with indicia 637 on the knob 632, to provide a user with information about the extent of extension of the stylet 612. In some embodiments, the handle 630 comprises sensors configured to communicate extension information with a nerve modulation system.

In some embodiments, the handle 630 comprises a rotatable knob. Gearing (e.g., a worm gear, bevel gears, rack and pinion, etc.) can be used to translate the rotation of the knob into longitudinal movement of the stylet 612. The handle may comprise detents (e.g., providing audible and/or tactile feedback), indicia, sensors, etc. to provide a user with information about the extent of extension of the stylet 612. In some embodiments, the knob 632 may be longitudinally advanced to provide rough or gross motion and the knob 632 may be rotated to provide fine motion.

In some embodiments, the handle 630 is rotatable relative to the needle body 602. Gearing (e.g., a worm gear, bevel gears, rack and pinion, etc.) can be used to translate the rotation of the handle 630 into longitudinal movement of the stylet 612. The handle may comprise detents, indicia, sensors, etc. to provide a user with information about the extent of extension of the stylet 612.

The stylet 612 may be replaceable, for example by being removably coupled to the handle 630. If the stylet 612 is bent beyond a useful tolerance, the existing stylet 612 can be removed and a second stylet 612 can be inserted into the needle body 602 and coupled to the handle 630. The stylet 612, the handle 630, and/or the needle body 602 may comprise keyed or alignment features to ensure that the second stylet 612 exits the aperture 607 along a curved path upon longitudinal movement.

FIG. 6B schematically illustrates an example delivery device 640 for a treatment device. The delivery device 640 comprises a catheter 642 comprising a first lumen 644 and a second lumen 646. An imaging device (e.g., an optical scope, an ultrasound scanner, etc.) can be advanced through the first lumen 644 to a treatment site. The lumen 644 may be used to supply fluid (e.g., ultrasound fluid, saline, contrast, etc.) to the treatment site. The imaging device may be used to image the treatment site. A treatment device (e.g., the treatment device 600 and/or other treatment devices) can be advanced through the second lumen 646 to the treatment site. The imaging device may be used to image the treatment device. A system may comprise the treatment device 600 and the delivery device 640. The delivery device 640 may be configured to rest on the surface of a subject (e.g., to not penetrate the skin of a subject). In certain such embodiments, the lumens 644, 646 may help to position the imaging device and the treatment device in a particular orientation, spacing, etc.

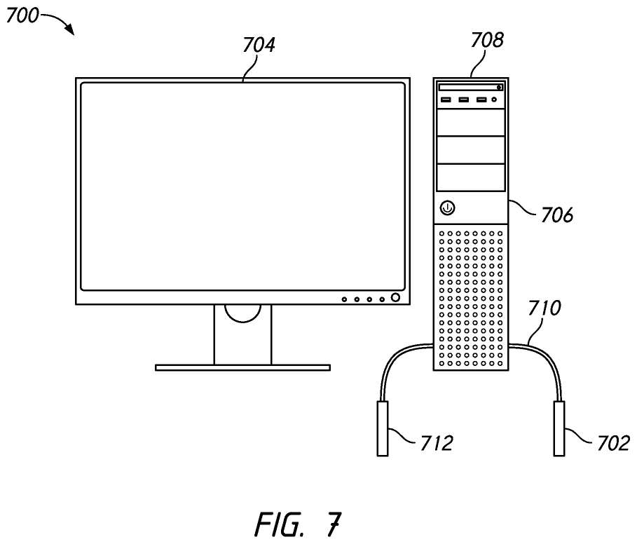

FIG. 7 is a schematic diagram of an example tissue treatment (e.g., nerve modulation) system 700. The system 700 serves as the user interface and provides the energy to a treatment device 702. The system 700 includes a display screen 704, energy generator 706, a control computer 708, and a removable connector 710 between the control computer 706 and the treatment device 702. The system 700 optionally comprises an imaging device 712. The display screen 702 may be a touch screen. The system 700 may comprise other inputs (e.g., a mouse, a keyboard, a track ball, foot pedal, etc.). In some embodiments, the control computer 708 may comprise the energy generator 706 or vice versa, or the energy generator 706 and the control computer 708 may be integral. Referring again to FIG. 6B, the imaging device 712 may be configured to be advanced through the first lumen 654 and the treatment device 702 is configured to be advanced through the second lumen 606. A system may comprise the system 700, the treatment device 600, and the delivery device 650. A system may comprise the system 700 and the treatment device 600. A system may comprise the system 700 and the delivery device 650. The treatment device 702 may comprise one or more electrodes configured to generate radiofrequency (RF) energy to ablate tissue around the one or more electrodes. The RF may be monopolar or bipolar. The imaging device 712 may comprise a laparoscope, an ultrasound imager, etc. The display screen 702 may show images from the imaging device 712. The images may be manually and/or automatically annotated, for example to mark anatomical landmarks, show an expected ablation area, etc.

The treatment device may be configured to apply bipolar RF energy to an anode and a cathode at a frequency between about 100 kilohertz (kHz) and about 100 megahertz (MHz) (e.g., about 100 kHz, about 250 kHz, about 450 kHz, about 500 kHz, about 550 kHz, about 1 MHz, about 10 MHz, about 50 MHz, about 100 MHz, and ranges between such values), a power between about 0.1 Watts (W) and about 100 W (e.g., about 0.1 W, about 1 W, about 5 W, about 10 W, about 15 W, about 20 W, about 25 W, about 50 W, about 75 W, about 100 W, and ranges between such values), a current between about 0.5 milliamperes (mA) and about 5 amperes (A) (e.g., about 0.5 mA, about 1 mA, about 10 mA, about 100 mA, about 500 mA, about 1 A, about 1.5 A, about 2 A, about 3 A, about 4 A, about 5 A, and ranges between such values) (if measured as root mean squared (rms), a current between about 0.25 mAᵣₘₛ and about 3 Aᵣₘₛ (e.g., about 0.25 mAᵣₘₛ, about 0.5 mAᵣₘₛ, about 1 mAᵣₘₛ, about 10 mAᵣₘₛ, about 100 mAᵣₘₛ, about 500 mAᵣₘₛ, about 750 mAᵣₘₛ, about 1 Arm, about 1.5 Aᵣₘₛ, about 2 Arm, about 2.5 Aᵣₘₛ, about 3 Aᵣₘₛ, and ranges between such values), a duration between about 1 second (s) and about 20 minutes (min) (e.g., about 1 s, about 3 s, about 5 s, about 10 s, about 15 s, about 20 s, about 30 s, about 45 s, about 1 min, about 2 min, about 3 min, about 4 min, about 5 min, about 10 min, about 15 min, about 20 min, and ranges between such values), etc. For example, a non-limiting example treatment energy may have a frequency between about 450 kHz and about 500 kHz, a power between about 10 W and about 20 W, a current between about 1 A and about 2 A, and a duration between about 30 s and about 5 min. Stimulation may be pulsed for a portion of the duration (e.g., a few seconds at a time) and turned off or not applied for other portions of the duration. Other parameters, for example depending on the specific treatment device 702, are also possible.

The treatment device 600 can be used to perform one ablation or a plurality of ablations. The treatment device 600 may be sterilizable for use on a plurality of subjects. The treatment device 600 may comprise electronic circuitry 631 schematically shown in FIG. 6A as being in the handle 630, but the electronic circuitry 631 can be located at any suitable position on the treatment device 600. The electronic circuitry 631 may be configured to track use information (e.g., the number of uses of the treatment device 600, the number of times the treatment device 600 is sterilized (indicative of the number of procedures) (e.g., using thermocouple information to sense high temperature, time between treatments, etc.), performance data (e.g., duration of use), defects, power used, etc.), which can be used for billing users by the number of uses. For example, whenever the treatment device 600 is coupled to a system 700 (e.g., by connecting the treatment device 600 to the connector 710), the use information may be transmitted locally and/or over a network where a supplier can access the information for billing purposes. In some embodiments, the connector 710 may comprise the electronic circuitry 631. In certain such embodiments, upon connection of the treatment device 702 to the connector 710, a usage may be measured. The electronic circuitry 631 may derive power from the system 700. The electronic circuitry 631 may comprise, for example, a processor and/or memory. In embodiments in which the treatment device 600 comprises the electronic circuitry 631, the electronic circuitry 631 may comprise a power source (e.g., for detecting information when not coupled to the system 700). The components of the electronic circuitry 631 may be configured to withstand sterilization. The electronic circuitry 631 may comprise insulation to at least partially shield the electrical components of the electronic circuitry 631 from sterilization.

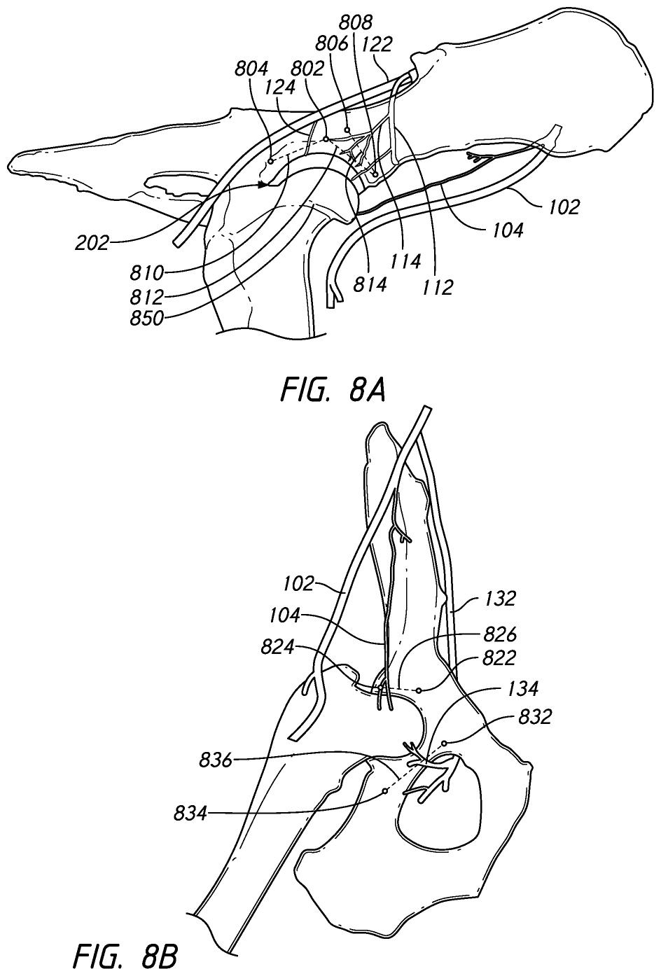

FIGS. 8A and 8B illustrate an example method of treating hip joint pain. The method comprises modulating (e.g., denervating, ablating, etc.) an articular branch 124 from the sciatic nerve 122 and an articular branch 114 from the cranial gluteal nerve 112. The method optionally comprises modulating an articular branch 104 from femoral nerve 102. The method optionally comprises modulating an articular branch 134 from the obturator nerve 132.

Using a dorsal and caudal approach, a user can insert a treatment device at a first point 802. The first point 802 may be an apex of a hip joint capsule. The user can advance the treatment device until the distal end contacts bone. Other approaches are also possible. Facets of the hip joint capsule 202 may be used to help determine a position of the first point 802. If the treatment device comprises the treatment device 600, for example, imaging can help to orient the treatment device 600 so that the stylet 612 is deployed until the second point 804. The acetabulum/acetabular rim (socket) and femoral head 850, for example, may be used to help determine a position of the second point 804. Imaging (e.g., fluoroscopy, ultrasound, etc.) may be used to verify the position of the treatment device prior to modulation. Terms such as “first,” second, “third,” etc. provide nomenclature that can help distinguish between different points or other multiple items discussed herein, and do not necessarily describe an order, a preference, a hierarchy, etc. For example, in some embodiments, the treatment device may be inserted at the second point 804 and extended to the first point 802. For example, the second point 804 may be easier to identify than the first point 802 when percutaneously positioning the treatment device. The acetabulum is bounded dorsally, cranially, and caudally by the acetabular rim. The targeted modulations may occur just above this rim when targeting the articular branch 124 of the sciatic nerve 122 and/or the articular branch 114 of the cranial gluteal nerve 112. Using borders (e.g., dorsal, cranial, caudal) of the hip joint or acetabulum can help to identify the rim, which may be termed the dorsal rim and/or the ventral rim). Although certain examples of insertion and extension points are provided herein, other insertion and extension points are also possible, for example others that would create a path that would include an articular branch nerve of interest. Bipolar radiofrequency energy can be applied between the first electrode 604 and the second electrode 614. The energy will extend along the path 810, forming a substantially linear or cylindrical ablation zone. The articular branch 124 from the sciatic nerve 122 lies in the path 810 such that the RF energy can modulate the articular branch 124. The stylet 612 can be retracted back into the needle body 602. After modulating the articular branch nerve 124, the pain is reduced. Pain reduction can be measured, for example, by walking evaluation, a biped station, rotation with external abduction, subluxation and iliopsoas, combinations thereof, a distraction index, etc. The test(s) can be performed before a procedure to establish a preoperative baseline, and then at one or more intervals after the procedure (e.g., one day, two days, one week, two weeks, one month, three months, six months, etc.). Imaging can also or alternatively be used to evaluate the tissue for signs of recovery.

In some embodiments, the user can rotate the treatment device 600 (e.g., between about 100° and about 160°) to orient the treatment device 600 so that the stylet 612 is deployed until the third point 808. Facets of the hip joint capsule 202 may be used to help determine a position of the third point 808. Imaging (e.g., fluoroscopy, ultrasound, etc.) may be used to verify the position of the treatment device prior to modulation. Bipolar radiofrequency energy can be applied between the first electrode 604 and the second electrode 614. The energy will extend along the path 812, forming a substantially linear or cylindrical ablation zone. The articular branch 114 from the cranial gluteal nerve 112 lies in the path 812 such that the RF energy can modulate the articular branch 114. After modulating the articular branch nerve 114, the pain is reduced. The stylet 612 can be retracted back into the needle body 602. Rotating the treatment device 600 between energy application can reduce the number of puncture sites, which can reduce soft tissue trauma and a number of possible infection sites. A smaller amount of rotation is also possible, for example to make sure that the first tissue was treated enough to capture the articular branch nerve.

In some embodiments, using a dorsal and caudal approach, the user can insert the treatment device at a fourth point 806. Other approaches are also possible. The user can advance the treatment device until the distal end contacts bone. If the treatment device comprises the treatment device 600, for example, imaging can help to orient the treatment device 600 so that the stylet 612 is deployed until the third point 808. Imaging (e.g., fluoroscopy, ultrasound, etc.) may be used to verify the position of the treatment device prior to modulation. In some embodiments, the treatment device may be inserted at the third point 808 and extended to the fourth point 808. Bipolar radiofrequency energy can be applied between the first electrode 604 and the second electrode 614. The energy will extend along the path 814, forming a substantially linear or cylindrical ablation zone. The articular branch 114 from the cranial gluteal nerve 112 lies in the path 812 such that the RF energy can modulate the articular branch 114. After modulating the articular branch nerve 114, the pain is reduced. The stylet 612 can be retracted back into the needle body 602. Removing and then reinserting the treatment device can provide greater flexibility in treatment, for example if extension from the first point 802 to the second point 804 is good for modulating the articular branch 124 but the anatomy makes extension from the first point 802 to the third point 808 difficult for modulating the articular branch 114. In some embodiments, a second puncture site can be useful for targeting a plurality of articular branches 114 (e.g., as show in FIG. 8A). In some embodiments, a second puncture site can be useful when the stylet 612 is extended from an edge towards a center. In some embodiments, both rotation and an additional puncture site may be used.

The lengths of the paths 810, 812, 814 are preferably long enough to capture an articular branch to be modulated but short enough that the modulation energy can be somewhat targeted. One, some, or all of the paths 810, 812, 814 may be along a portion of the diameter of the hip joint, for example between about 1/10 and about ⅔ (e.g., about 1/10, about ⅛, about ⅙, about ¼, about ⅓, about ½, about ⅔, and ranges between such values). For example, in an average adult German Shepherd, the acetabulum has a diameter of about 20 mm, so one, some, or all of the paths 810, 812, 814 may have a length between about 2 mm and about 13.3 mm (e.g., about 2 mm, about 2.5 mm, about 3.3 mm, about 5 mm, about 6.7 mm, about 10 mm, about 13.3 mm, and ranges between such values). For another example, in an average adult horse, the acetabulum has a diameter of about 56 mm, so one, some, or all of the paths 810, 812, 814 may have a length between about 5 mm and about 37 mm (e.g., about 5 mm, about 6 mm, about 7 mm, about 9 mm, about 14 mm, about 19 mm, about 28 mm, about 37 mm, and ranges between such values). For another example, in an average adult human male, the acetabulum has a diameter of about 52 mm, so one, some, or all of the paths 810, 812, 814 may have a length between about 5 mm and about 35 mm (e.g., about 5 mm, about 6 mm, about 7 mm, about 9 mm, about 13 mm, about 17 mm, about 26 mm, about 35 mm, and ranges between such values). For another example, in an average adult human female, the acetabulum has a diameter of about 46 mm, so one, some, or all of the paths 810, 812, 814 may have a length between about 4 mm and about 31 mm (e.g., about 4 mm, about 5 mm, about 6 mm, about 8 mm, about 12 mm, about 15 mm, about 23 mm, about 31 mm, and ranges between such values). Larger breeds or species (e.g., bovine, equine, large feline, pachyderm) may have longer paths 810, 812, 814. Smaller breeds or species (e.g., feline) may have shorter paths 810, 812, 814. The stylet 612 may be configured to treat both large and small breeds of one type of species. The stylet 612 may be configured to treat both large and small breeds and species.

When the modulation is close to the surface of the bone and/or follows a substantially linear path, tissue such as muscles, tendons, blood vessels, etc. can be substantially avoided. For example, RF ablation around a muscle can cause muscle atrophy, but RF ablation distant to the muscles can avoid causing muscle atrophy.

Referring to FIG. 8B, additional articular branches 104 and/or 134 can be optionally modulated. Using a dorsal and caudal, a true lateral, a cranial and ventral, etc. approach a user can insert a treatment device at a fifth point 822. Other approaches are also possible. The user can advance the treatment device until the distal end contacts bone. Facets of the hip joint capsule, the acetabular notch, blood vessels, and/or other anatomical landmarks may be used to help determine a position of the fifth point 822. If the treatment device comprises the treatment device 600, for example, imaging can help to orient the treatment device 600 so that the stylet 612 is deployed until the sixth point 824. Facets of the hip joint capsule, blood vessels, and/or other anatomical landmarks may be used to help determine a position of the sixth point 824. Imaging (e.g., fluoroscopy, ultrasound, etc.) may be used to verify the position of the treatment device prior to modulation. Bipolar radiofrequency energy can be applied between the first electrode 604 and the second electrode 614. The energy will extend along the path 826, forming a substantially linear or cylindrical ablation zone. The articular branch 104 from the femoral nerve 102 lies in the path 826 such that the RF energy can modulate the articular branch 104. The stylet 612 can be retracted back into the needle body 602. After modulating the articular branch nerve 104, the pain is reduced.

Using a ventral and caudal approach, user can insert a treatment device at a seventh point 832. Other approaches are also possible. In some embodiments, the limb of the hip joint may be lifted to increase access. The user can advance the treatment device until the distal end contacts bone. Facets of the hip joint capsule, the obturator foramen, the acetabular notch, blood vessels, and/or other anatomical landmarks may be used to help determine a position of the seventh point 832. If the treatment device comprises the treatment device 600, for example, imaging can help to orient the treatment device 600 so that the stylet 612 is deployed until the eighth point 834. Facets of the hip joint capsule, blood vessels, and/or other anatomical landmarks may be used to help determine a position of the eighth point 834. Imaging (e.g., fluoroscopy, ultrasound, etc.) may be used to verify the position of the treatment device prior to modulation. Bipolar radiofrequency energy can be applied between the first electrode 604 and the second electrode 614. The energy will extend along the path 836, forming a substantially linear or cylindrical ablation zone. The articular branch 134 from the obturator nerve 132 lies in the path 836 such that the RF energy can modulate the articular branch 134. The stylet 612 can be retracted back into the needle body 602. After modulating the articular branch nerve 134, the pain is reduced.

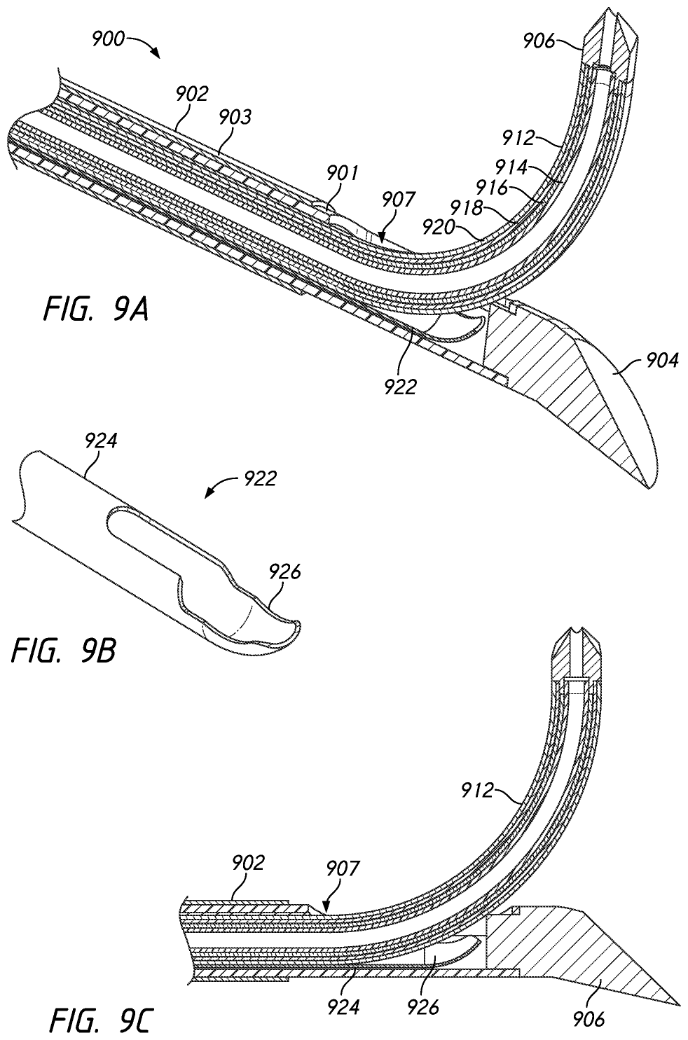

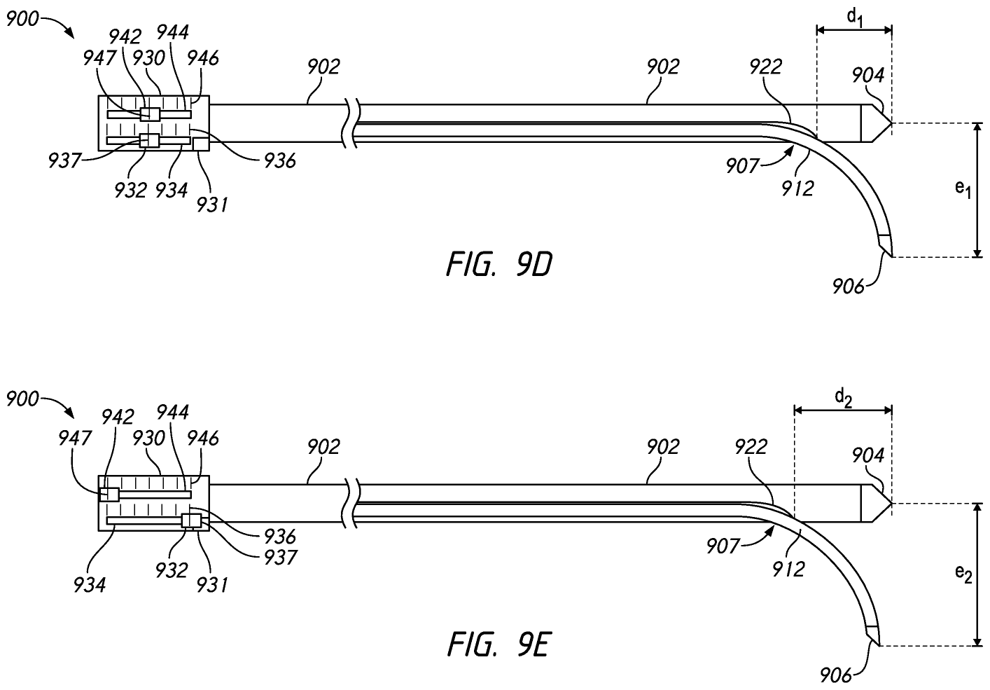

FIG. 9A is a partial cross-sectional perspective view of an example tissue treatment device 900. The device 900 may share features with the device 600 and/or other tissue treatment devices herein (e.g., radiopaque markers, echogenic surfaces, electrode type, tip type, sensors, materials, dimensions including distances, usage, handle features, connection to a tissue treatment (e.g., nerve modulation (e.g., ablation)) system, etc.). The treatment device 900 comprises a needle body 902. The needle body 902 may comprise a shaft 901 and an outer tube 903. The outer tube 903 may comprise a heat shrink material. The distal end of the needle body 902 comprises a tissue-penetrating tip 904. The tip 904 comprises a first electrode. The portion of the needle body 902 proximal to the tip 904 may comprise electrically insulating material. The needle body 902 comprises an aperture 907. The needle body 902 may comprise a longitudinally-extending lumen in communication with the aperture 907. The needle body 902 may be hollow at least proximal to the aperture 907.

The treatment device 900 comprises a stylet 912 configured to exit the aperture 907 and curve radially away from the needle body 902. The stylet 912 may comprise a plurality of layers or tubes. For example, the stylet 912 shown in FIG. 9A includes a first tube 914, a second tube 916, a third tube 918, and a fourth tube 920. The first tube 914 may comprise a shape set shape memory tube, for example configured to take a curved shape when not confined by the needle body 902. The second tube 916 may comprise a heat shrink. The third tube 918 may comprise a shape set and laser cut shape memory tube, for example configured to take a curved shape when not confined by the needle body 902. The third tube 918 in combination with the first tube 914 can provide refinement of the shape, additional stiffness, and/or a secondary pathway for return if desired. The fourth tube 920 may comprise a heat shrink. For example, the fourth tube may at least partially fill or cover features cut into the third tube 918. The distal end of the stylet 912 comprises a tissue-penetrating tip 906. The tip 906 comprises a second electrode. More or fewer tubes are also possible. For example, the first tube 914 or the third tube 918 may be omitted. The second electrode may be electrically coupled to a signal generator by, for example, the first tube 914 and/or the third tube 918. Bipolar radiofrequency signals can be applied between the first electrode and the second electrode. In some embodiments, the first electrode and the second electrode can be separate from the tips 904, 906, for example as described with respect to the device 600.

The device 900 comprises a ramp 922. FIG. 9B is a partial perspective view of an example component, specifically the ramp 922, of the tissue treatment device 900 of FIG. 9A. FIG. 9C is a partial cross-sectional side view of the tissue treatment device 900 of FIG. 9A. The ramp 922 comprises an elongate section 924 and a deflection section 926. The elongate section 924 is coupled to an actuator in a handle that is configured to longitudinally move the ramp 922. The elongate section 924 may comprise a tube (e.g., as shown in FIG. 9B), a wire, a flat segment, etc. The deflection section 926 is configured to direct the stylet 912 laterally out of the aperture 907 of the needle body 902. The deflection section 926 may be curved (e.g., comprising a continuous curve as shown in FIGS. 9A-9C, comprising a plurality of arcuate segments), straight (e.g., at an angle), combinations thereof, and the like. The ramp 922 is longitudinally movable in the needle body 902.

FIG. 9D is a schematic diagram of the tissue treatment device 900 of FIG. 9A. The device 900 comprises a handle 930. The handle 930 may share features with the handle 630 (e.g., electronic circuitry 931, gearing, etc.). The handle 930 comprises a first knob 932 configured to slide in a first channel 934 and a second knob 944 configured to slide in a second channel 944. Distally advancing the second knob 942 distally advances the stylet 912. The handle 930 comprises indicia 946, for example alignable with indicia 947 on the knob 942, to provide a user with information about the extent of extension of the stylet 912. Distally retracting the first knob 932 distally retracts the ramp 922. The handle 930 comprises indicia 936, for example alignable with indicia 937 on the knob 932, to provide a user with information about the extent of retraction and/or longitudinal position of the ramp 922. In some implementations, the channel 934 approximates the aperture 907 (e.g., the position of the knob 932 in the channel 934 approximates the position of the ramp 922 in the aperture 907).

The position of the ramp 922 and the extent of deployment of the stylet 912 can influence the position of the second electrode of the stylet 912 (e.g., the tip 906 or a separate electrode structure). For example, in FIG. 9D, the knob 942 is retracted about half way through the channel 944 such that the distal tip of the ramp 922 is a first distance d₁ from the distal tip of the tip 904, and the knob 932 is advanced about half way through the channel 934 such that the distal tip of the tip 906 of the stylet 912 is a first lateral distance e₁ from the distal tip of the tip 904, the longitudinal axis of the needle body 902, or the like. Applying a bipolar radiofrequency signal between the tip 904 and tip 906 will create an approximately linear lesion between the tip 904 and the tip 906. If the tip 904 is proximate to a bone and the tip 906 is in the position illustrated in FIG. 9D, the lesion will be proximate to the bone.

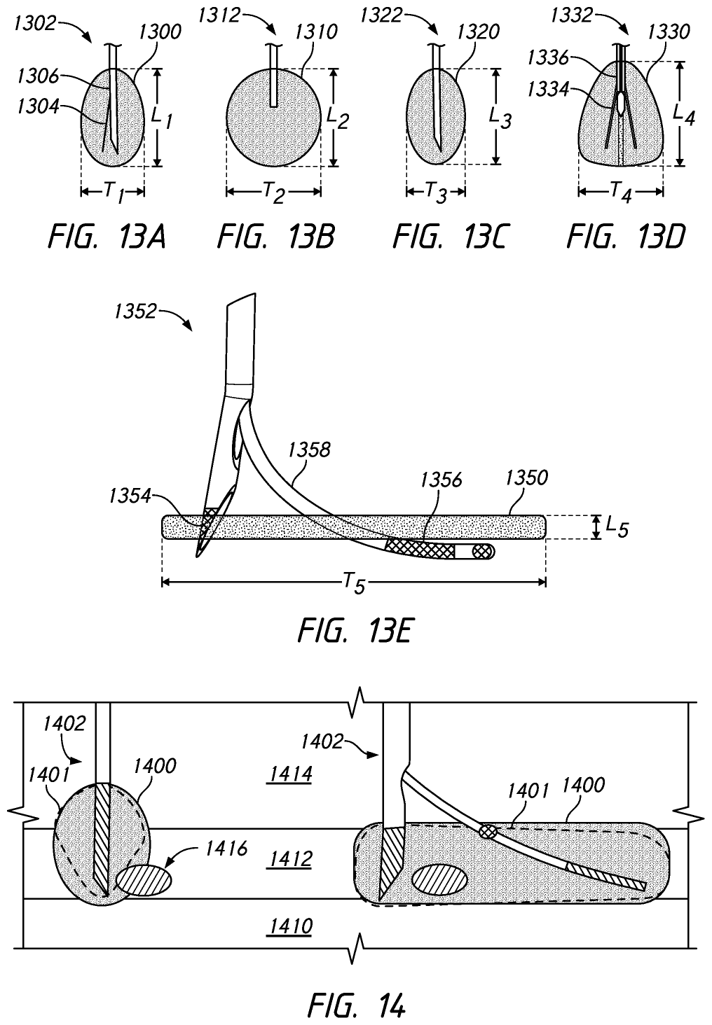

FIG. 9E is another schematic diagram of the tissue treatment device 900 of FIG. 9A. In some embodiments, the user may desire to take a different angle of deployment of the stylet 912, for example due to the tissue around the treatment site. Adjusting where the stylet 912 comes out the side of the needle body 902 can affect the size of the ablation (e.g. thickness T in FIG. 13E). For example, if the stylet 912 comes out more proximally, the thickness T will be relatively larger, and if the stylet 912 comes out more distally, the thickness T will be relatively smaller. Adjusting where the stylet 912 comes out of the side of the needle body 902 can ensure that the second electrode makes it around the curvature of the joint surface. For example, if the needle body 902 is docked on the dorsal aspect (12:00) on the acetabular rim (e.g. dorsal rim), the surface is concave. The position of the ramp 922 and the extent of deployment of the stylet 912 can again influence the position of the second electrode of the stylet 912 (e.g., the tip 906 or a separate electrode structure). For example, in FIG. 9E, the knob 942 is fully retracted through the channel 944 such that the distal tip of the ramp 922 is a second distance d₂ (greater than the first distance d₁) from the distal tip of the tip 904, and the knob 932 is fully advanced through the channel 934 such that the distal tip of the tip 906 of the stylet 912 is a second lateral distance e₂ (greater than the first lateral distance e₁) from the distal tip of the tip 904, the longitudinal axis of the needle body 902, or the like. Even though the stylet 912 is advanced from the needle body 902 a greater longitudinal distance and a greater lateral distance, the tip 906 is still proximate a same longitudinal position as the tip 904, which can both be proximate a bone, for example.

In some embodiments, the knobs 932, 942 can be mechanically linked. For example, if the treatment method preferably results in the tip 906 being proximate a same longitudinal position as the tip 904, the handle 930 can include gears, stop surfaces, etc. such that proximal retraction of the knob 942 affects the permitted extent of advancement of the knob 932. In some implementations, the indicia 936, 946 can be color coded. For example, if the ramp 922 is in a proximal-most position, then the stylet 912 is preferably fully advanced out of the aperture 907, so the proximal-most indicia 946 and the distal-most indicia 936 can be the same color to alert the user that matching colors means proper positioning.

The ramp 922 preferably moves longitudinally back and forth. For example, in some embodiments, the ramp 922 does not move laterally (e.g., out of the needle body 902). The ramp 922 preferably is rigid or otherwise maintains its shape. For example, in some embodiments, the deflection surface 926 does not deform, tilt, etc. to change an angle of deflection. The ramp 922 preferably comprises an open deflection surface. For example, in some embodiments, the deflection surface 926 does not comprise a fully annular tube. The ramp 922 is preferably in the needle body 902. For example, in some embodiments, the device 900 does not comprise a tubular that interacts with the needle body 902 to change the aperture 907 or the like.

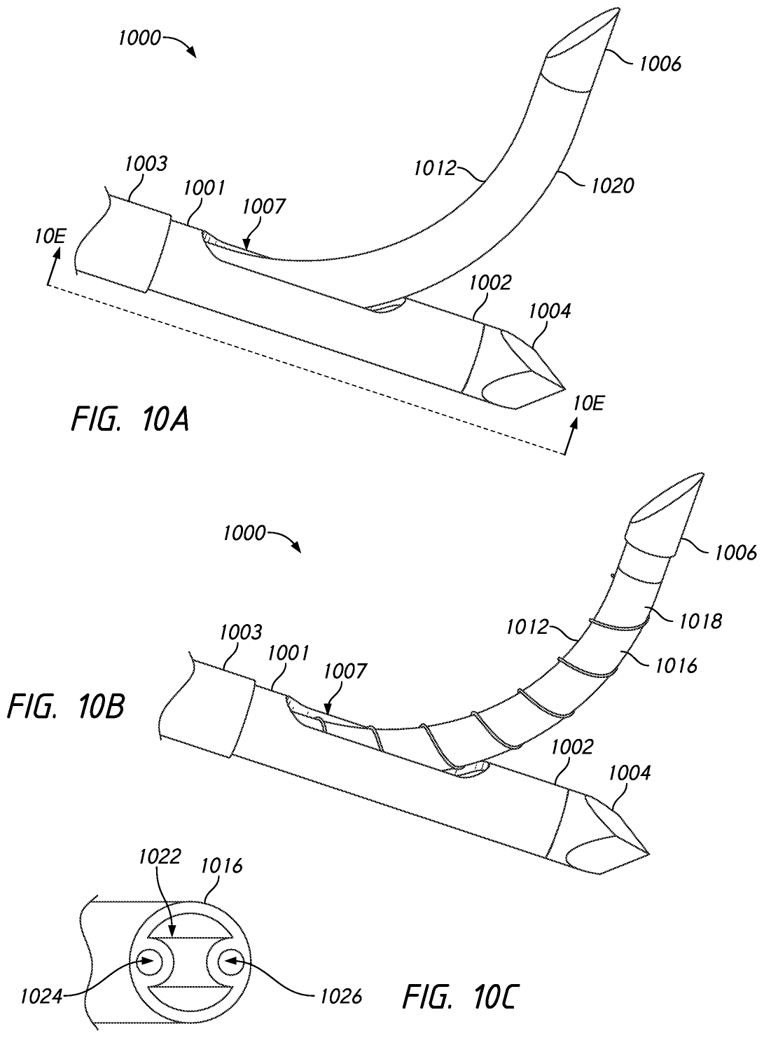

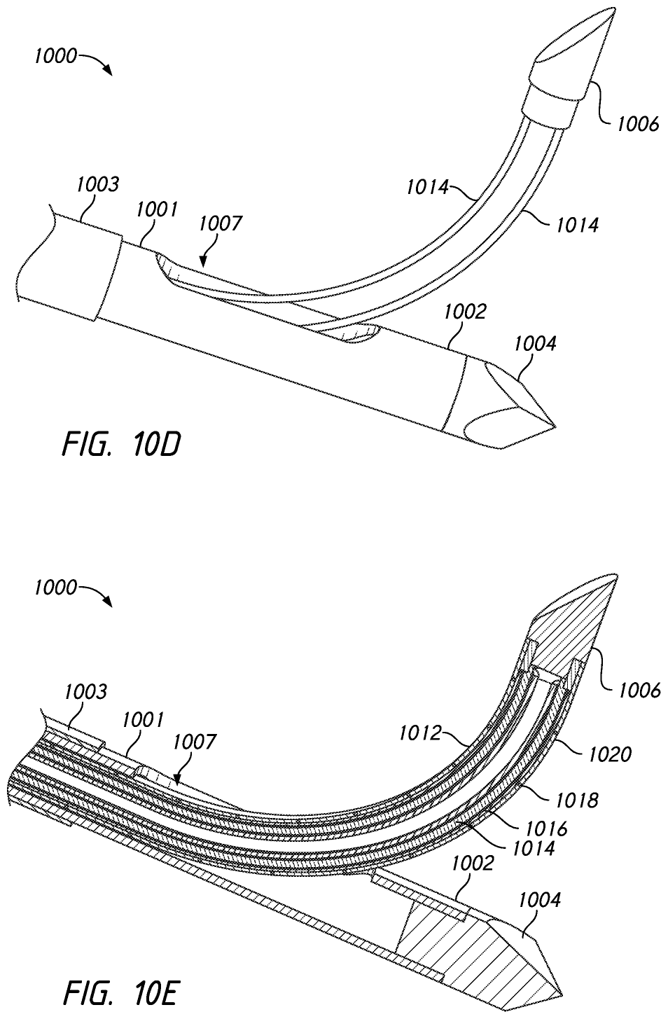

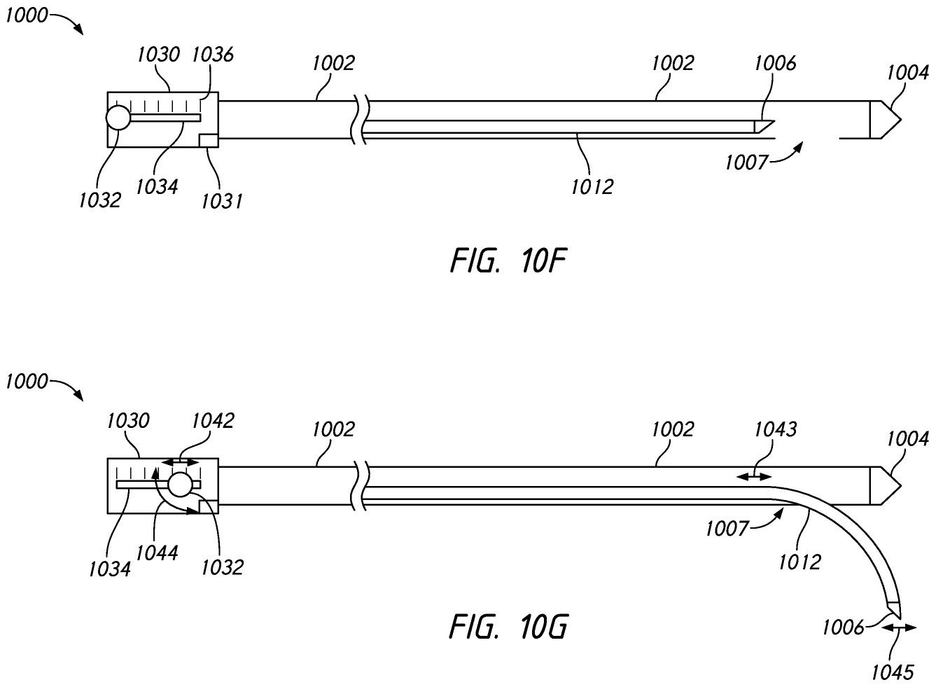

FIG. 10A is a partial perspective view of an example tissue treatment device 1000. The device 1000 may share features with the device 600, the device 900, and/or other tissue treatment devices herein (e.g., radiopaque markers, echogenic surfaces, electrode type, tip type, sensors, materials, dimensions including distances, usage, handle features, connection to a tissue treatment (e.g., nerve modulation (e.g., ablation)) system, movable ramp, etc.). The treatment device 1000 comprises a needle body 1002. The needle body 1002 may comprise a shaft 1001 and an outer tube 1003. The outer tube 1003 may comprise a heat shrink material. The distal end of the needle body 1002 comprises a tissue-penetrating tip 1004. The tip 1004 comprises a first electrode. The portion of the needle body 1002 proximal to the tip 1004 may comprise electrically insulating material. The needle body 1002 comprises an aperture 1007. The needle body 1002 may comprise a longitudinally-extending lumen in communication with the aperture 1007. The needle body 1002 may be hollow at least proximal to the aperture 1007.

The treatment device 1000 comprises a stylet 1012 configured to exit the aperture 1007 and curve radially away from the needle body 1002. The stylet 1012 may comprise a plurality of layers or tubes or components. For example, FIG. 10A shows the outer tube 1020 of the stylet 1012. The outer tube 1020 may comprise a heat shrink. The outer tube 1020 may comprise a polymer such as polyether ether ketone (PEEK), polytetrafluoroethylene (PTFE), combinations thereof, or the like. The distal end of the stylet 1012 comprises a tissue-penetrating tip 1006. The tip 1006 comprises a second electrode.

FIG. 10B is a partial perspective view of the tissue treatment device 1000 of FIG. 10A with a component, specifically the stylet outer tube 1020, removed. FIG. 10B shows a catheter 1016 and an optional stiffener 1018. The stiffener 1018 may comprise a coil (e.g., as shown in FIG. 10B), a braid, a tube, combinations thereof, or the like. FIG. 10C is a partial cross-sectional distal end view of an example component, specifically the catheter 1016, of the tissue treatment device 1000 of FIG. 10A. The catheter 1016 comprises a first lumen 1024 configured to house a pull wire. The catheter 1016 optionally comprises a second lumen 1026 configured to house a second pull wire. The first lumen 1024 may be circumferentially opposite (e.g., circumferentially spaced by 180°) the second lumen 1026. The first lumen 1024 may have a diameter between about 0.005 inches (in.) (approx. 0.13 mm) and about 0.02 in. (approx. 0.51 mm) (e.g., about 0.005 in. (approx. 0.13 mm), about 0.008 in. (approx. 0.2 mm), about 0.011 in. (approx. 0.28 mm), about 0.014 in. (approx. 0.36 mm), about 0.017 in. (approx. 0.43 mm), about 0.02 in. (approx. 0.51 mm), ranges between such values, and the like). The catheter 1016 optionally comprises a central lumen 1022. The central lumen 1022 may take a shape remaining after the lumen 1024 and optionally the lumen 1026 are removed (e.g., as shown in FIG. 10C). The central lumen 1022 may have a circular, oval, elliptical, etc. lateral cross section. The catheter 1016 may comprise a polymer such as PEEK, PTFE, combinations thereof, or the like. The catheter 1016 may have an outer diameter between about 2 French (Fr) (approx. 0.026 in.; approx. 0.66 mm) and about 6 Fr (approx. 0.078 in.; approx. 2 mm) (e.g., about 2 Fr (approx. 0.026 in.; approx. 0.66 mm), about 3 Fr (approx. 0.039 in.; approx. 1 mm), about 4 Fr (approx. 0.052 in.; approx. 1.3 mm), about 5 Fr (approx. 0.065 in.; approx. 1.6 mm), about 6 Fr (approx. 0.078 in.; approx. 2 mm), ranges between such values, and the like).

FIG. 10D is a partial perspective view of the tissue treatment device 1000 of FIG. 10A with additional components, specifically the stiffener 1018 and the catheter 1016, removed. FIG. 10D shows pull wires 1014. The pull wires 1014 extend through the lumens 1024, 1026. The pull wires 1014 are configured to deflect the stylet 1012 proximally and distally. For example, if the pull wire 1014 connected to an upper side of the tip 1004 (left as illustrated in FIG. 10D) is proximally retracted, the tip 1006 will deflect proximally, and if the pull wire 1014 connected to a lower side of the tip 1004 (right as illustrated in FIG. 10D) is proximally retracted, the tip 1006 will deflect distally. Preferably, the stylet 1012 only deflects in one or two directions along a plane including the longitudinal axis of the needle body 1002. One or both of the pull wires 1014 can provide electrical signals to the tip 1006. In some embodiments, a separate wire or other electrical conductor (e.g., extending through the central lumen 1022) provides electrical signals to the tip 1006.

FIG. 10E is a partial cross-sectional side view of the tissue treatment device 1000 of FIG. 10A along the line 10E-10E. FIG. 10E shows the components of the stylet 1012 in combination. For example, FIG. 10E shows the pull wires 1014 extending through the lumens 1024, 1026 of the catheter 1016 and coupled to the tip 1006.

FIG. 10F is a schematic diagram of the tissue treatment device 1000 of FIG. 10A. The device 1000 comprises a handle 1030. The handle 1030 may share features with the handle 630, 930 (e.g., electronic circuitry 1031, gearing, etc.). The handle 1030 comprises a knob 1032 configured to slide in a channel 1034 and to rotate about an axis perpendicular to the channel 1034. Distally advancing the knob 1032 distally advances the stylet 1012. The handle 1030 comprises indicia 1036 to provide a user with information about the extent of extension of the stylet 1012. Rotating the knob 1032 in a first direction (e.g., clockwise) deflects the stylet 1012 in a first direction (e.g., proximally) and rotating the knob 1032 in a second direction (e.g., counterclockwise) deflects the stylet 1012 in a second direction (e.g., distally). By rotating the knob 1032 and moving the knob 1032 through the channel 1034, a user can effectively steer the stylet 1012. Steering the stylet 1012 can help to make sure that the tip 1006 is close to bone and does not deflect upwardly into muscle, for example, upon additional extension due in contrast to a stylet having an accurate shape set that might begin to curve away from bone as the stylet is further extended. In some anatomy, the stylet 1012 may desirable deflect distally to be able to remain close to bone. Steering the stylet 1012 can help to avoid crucial structures. Steering the stylet 1012 can allow for poor initial placement or variances in anatomy from patient to patient (e.g., initial placement may be the same for every case, but variances in anatomy such as joint shape, size, etc. and whether congenital or due to disease may use a change in direction to navigate to desired end point. The acetabulum and most joint surfaces are curved/concave. The adjustability provided by a steerable stylet 1012 can allow a user to articulate/steer the electrode around curved structures. Steering the stylet 1012 can allow the tip 1006 to be navigated to a desired position that the stylet 1012 may not otherwise achieve upon deployment. For example, the tip 1006 may be deflected to traverse along a bone for a desired extension distance, whereas a non-steered stylet may start to curve away from bone after some extension distance. Steering the stylet 1012 can allow the stylet 1012 to avoid certain tissue. For example, the stylet may be deflected to avoid tendons, cartilage, etc. that a non-steered stylet may damage.

FIG. 10G is another schematic diagram of the tissue treatment device 1000 of FIG. 10A. FIG. 10G shows the knob 1032 moving through the channel 1034, as indicated by the arrow 1042 and the corresponding arrow 1043, and being rotated, as shown by the arrow 1044 and the corresponding arrow 1045, which shows the deflection upon rotation of the knob 1032. Other mechanisms are also possible. For example, the knob 1032 could comprise an annular element extending around the handle 1030. For another example, the knob 1032 could comprise a lever configured to tilt proximally and distally to deflect the stylet 1012 proximally and distally.

In some embodiments, the knob 1032 can be mechanically geared to couple the rotation and the position along the channel 1034. For example, the knob 1032 may be configured so that rotation of the knob 1032 has a greater effect when the knob 1032 is further distally advanced than when the knob 1032 is less distally advanced.

The steerable stylet 1012 preferably extends out of a lateral side of the needle body 1002. For example, in some embodiments, the stylet 1012 does not exit or extend out of a distal end of the needle body 1002. The steerable stylet 1012 preferably is configured to navigate through soft tissue. For example, in some embodiments, the stylet 1012 cannot penetrate bone or navigate through spongy bone. The steerable stylet 1012 preferably includes an electrode that is used for neuromodulation (e.g., ablation), and thus should not be confused with a guidewire, a guide catheter, etc. In some embodiments, the steerable stylet 1012 is not a hollow steerable component that is then used to guide a separate electrode-bearing tube.

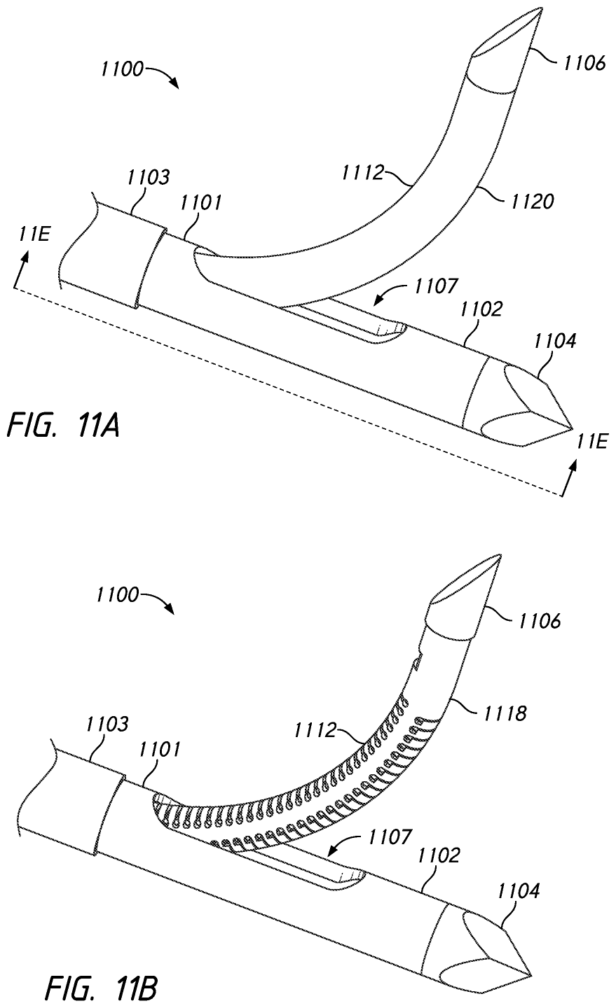

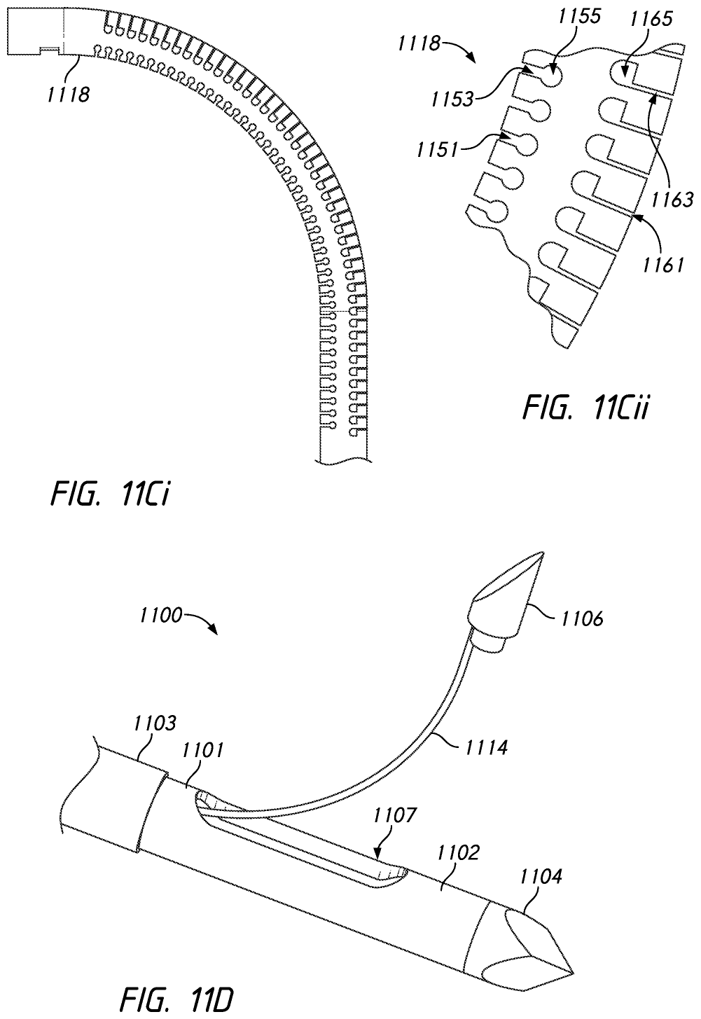

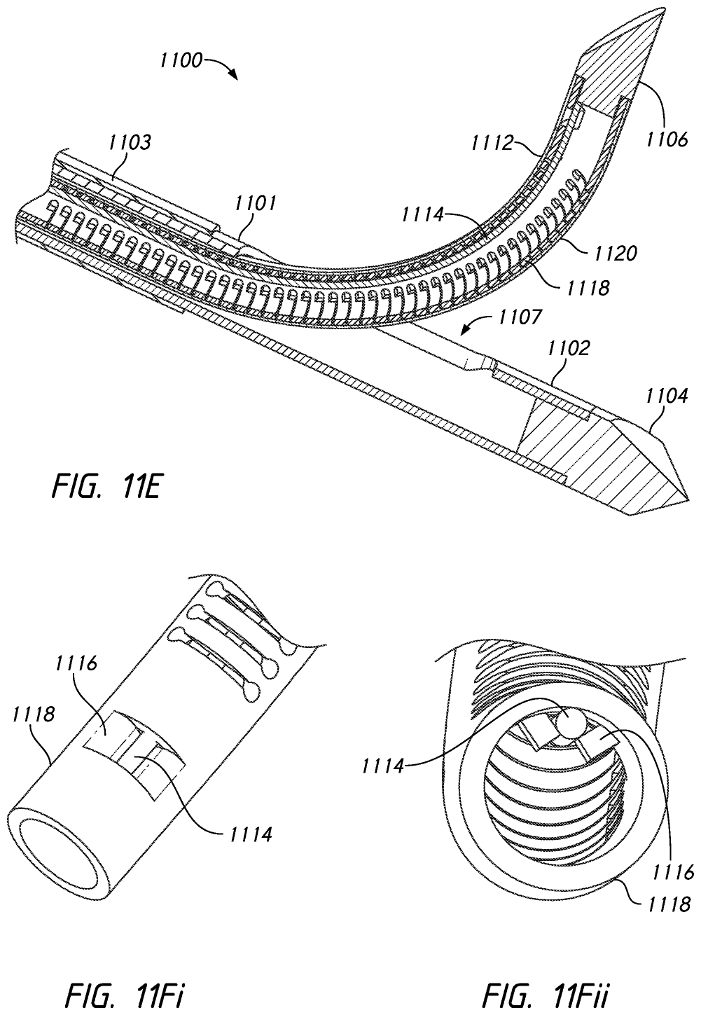

FIG. 11A is a partial perspective view of an example tissue treatment device 1100. The device 1100 may share features with the device 600, the device 900, the device 1000, and/or other tissue treatment devices herein (e.g., radiopaque markers, echogenic surfaces, electrode type, tip type, sensors, materials, dimensions including distances, usage, handle features, connection to a tissue treatment (e.g., nerve modulation (e.g., ablation)) system, movable ramp, etc.). The treatment device 1100 comprises a needle body 1102. The needle body 1102 may comprise a shaft 1101 and an outer tube 1103. The outer tube 1103 may comprise a heat shrink material. The distal end of the needle body 1102 comprises a tissue-penetrating tip 1104. The tip 1104 comprises a first electrode. The portion of the needle body 1102 proximal to the tip 1104 may comprise electrically insulating material. The needle body 1102 comprises an aperture 1107. The needle body 1102 may comprise a longitudinally-extending lumen in communication with the aperture 1107. The needle body 1102 may be hollow at least proximal to the aperture 1107.

The treatment device 1100 comprises a stylet 1112 configured to exit the aperture 1107 and curve radially away from the needle body 1102. The stylet 1112 may comprise a plurality of layers or tubes or components. For example, FIG. 11A shows the outer tube 1120 of the stylet 1112. The outer tube 1120 may comprise a heat shrink. The outer tube 1120 may comprise a polymer such PEEK, PTFE, combinations thereof, or the like. The outer tube 1120 may reduce damage to the needle body 1102 (e.g., from friction between the stylet 1112 and the needle body 1102 during deployment and retraction of the stylet 1112). The outer tube 1120 may have a thickness, for example, between about 0.002 in (approx. 0.05 mm) and about 0.02 in. (approx. 0.5 mm) (e.g., about 0.002 in (approx. 0.05 mm), about 0.005 in (approx. 0.13 mm), about 0.008 in (approx. 0.2 mm), about 0.01 in (approx. 0.25 mm), about 0.015 in (approx. 0.38 mm), about 0.02 in (approx. 0.5 mm), ranges between such values, and the like). The distal end of the stylet 1112 comprises a tissue-penetrating tip 1106. The tip 1106 comprises a second electrode.

FIG. 11B is a partial perspective view of the tissue treatment device 1100 of FIG. 11A with a component, specifically the stylet outer tube 1120, removed. FIG. 11B shows that the stylet 1112 comprises a tube 1118. The tube 1118 may comprise a coil, a braid, a tube, combinations thereof, or the like. The tube 1118 shown in FIG. 11B comprises a patterned (e.g., laser-cut) hypotube. The tube 1118 may be shape set (e.g., to a particular curvature). The tube 1118 may be not shape set. The tube 1118 may comprise, for example, stainless steel or other biocompatible materials. In some embodiments, the tube 1118 does not comprise a shape memory material such as nitinol.

FIG. 11Ci is a side view of an example component, the tube 1118, of the tissue treatment device 1100 of FIG. 11A. FIG. 11Cii is an expanded side view of the example component, the tube 1118, of FIG. 11Ci. The tube 1118 comprises a first plurality of kerfs 1151 and a second plurality of kerfs 1161. The first plurality of kerfs 1151 are on a first circumferential side of the tube 1118 and the second plurality of kerfs 1161 are on a second side of the tube 1118 opposite the first plurality of kerfs 1151. The pluralities of kerfs 1151, 1161 allow the tube 1118 to bend in one or two directions along a plane (e.g., the plane of the page with respect to FIG. 11Ci). The tube 1118 preferably does not bend in another direction during normal use of the device 1100. The kerfs 1151 may be the same as the kerfs 1161.

The kerfs 1151 may be different than the kerfs 1161 (e.g., as shown in FIGS. 11Ci and 11Cii). The kerfs 1151 may comprise a straight portion 1153 and an expanded portion 1155. The kerfs 1161 may comprise a straight portion 1163 and an expanded portion 1165. The straight portions 1153 may be thicker than the straight portions 1163. The straight portions 1153 may be shorter than the straight portions 1163. The expanded portions 1155 may be larger in area than the expanded portions 1165. The expanded portions 1155 may be a different shape than the expanded portions 1165. For example, FIG. 11Cii shows the expanded portions 1155 being circular and centered on the straight portions 1153, and the expanded portions 1165 being semicircular and having edges aligned with the straight portions 1151. The kerfs 1151 may have a first spacing and the kerfs 1161 may have a second spacing. The second spacing may be greater that the first spacing.

The differences in the kerfs 1151, 1161 can influence the bending of the tube 1118. For example, when the tube 1118 bends towards the kerfs 1151 (e.g., as shown in FIG. 11Cii), the lack of material in the wide straight portions 1153 can allow material to take the place of the space. Conversely, if the tube 1118 were to bend in the opposite direction, the lack of material in the narrow straight portions 1163 would allow less material to take the place of the space and impart less bending. For another example, when the tube 1118 bends towards the kerfs 1151 (e.g., as shown in FIG. 11Cii), the expanded portions 1155 can provide pivot points around along the same longitudinal axis as the straight portions 1153, and the expanded portions 1165 can provide pivot points that are on a different longitudinal axis as the straight portions 1163. These shapes and/or differentials can help to spread out the load and produce a device that is less prone to cracking and/or longer lasting. For another example, when the tube 1118 bends towards the kerfs 1151 (e.g., as shown in FIG. 11Cii), the kerfs 1151 being closely spaced can provide a reduced radius of curvature and the kerfs 1161 being more widely spaced can provide a higher radius of curvature, as desired for the bend.

The kerfs 1151, 1161 may be consistent over the substantial length of the tube 1118 (e.g., as shown in FIG. 11Ci). The kerfs 1151, 1161 may be vary over the length of the tube 1118 (e.g., having variable spacing, dimensions, and/or shapes, for example to make the tube 1118 more flexible towards the distal end). Other cut patterns are also possible. For example, the kerfs 1151, 1161 can be straight lines, I-lines, dumbbell lines, etc. For another example, kerfs can be formed from an interrupted helix. The outer tube 1120 may at least partially fill and/or cover kerfs in the tube 1118, for example to avoid pinching, inhibit liquid from flowing through the kerfs, etc. If the tube 1118 is conductive, the outer tube 1120 can electrically insulate the tube 1118.

FIG. 11D is a partial perspective view of the tissue treatment device 1100 of FIG. 11A with another component, specifically the tube 1118, removed. FIG. 11D shows a pull wire 1114. The pull wire 1114 extends through a lumen of the tube 1118. The pull wire is connected to a side of the tip 1106. The side is preferably the same side as the kerfs 1151. The pull wire 1114 is configured to deflect the stylet 1112 proximally. For example, if the pull wire 1114 is proximally retracted, the tip 1106 will deflect proximally. Preferably, the stylet 1112 only deflects in one or two directions along a plane including the longitudinal axis of the needle body 1102. In some embodiments, the tube 1118 is shape set (e.g., to be straight, to have a curve as shown in FIG. 11D, etc.). In some embodiments, the tube 1118 is not heat treated to impart a shape set, but still reverts towards a straight configuration in the absence of outside forces such as from the pull wire 1114. The pull wire 1114 can provide electrical signals to the tip 1106. In some embodiments, a separate wire, the tube 1118, or other electrical conductor (e.g., extending through the lumen of the tube 1118) provides electrical signals to the tip 1106.

FIG. 11E is a partial cross-sectional side view of the tissue treatment device 1100 of FIG. 11A along the line 11E-11E. FIG. 11E shows the components of the stylet 1112 in combination. For example, FIG. 11E shows the pull wire 1114 extending through the lumen of the tube 1118 and coupled to the tip 1106.

FIG. 11Fi is a partial side and distal end perspective view of example components, specifically the tube 1118 and the pull wire 1114, of the tissue treatment device 1100 of FIG. 11A. FIG. 11Fii is a partial distal end view of the example components, the tube 1118 and the pull wire 1114, of FIG. 11Ei. FIG. 11Fi shows an example implementation of optionally coupling the pull wire 1114 to the tube 1118. A distal segment of the tube 1118 is cut to have a “H” pattern, and then the material 1116 in the H is pressed radially inward. The pull tube 1114 is positioned between the inner surface of the tube 1118 and the formerly outer surface of the tube 1118 that is the pressed H material 1116. The material 1116 has a natural outward spring that bears against the pull wire 1114, holding the pull wire 1114 in place. In some embodiments, the pull wire 1114 may comprise a groove, detent, etc. configured to act with the material 1116. The distal end of the pull wire 1114 is preferably positioned distal to the H material 1116 so that the pull wire 1114 does not prolapse from the tube 1118. Cut shapes other than H can effect a same or similar result (e.g., E, S, U, V, parallel lines, combinations thereof, etc.). In some embodiments, the pull wire 1114 can also or alternatively be coupled to the tube 1118 by welding, soldering, adhesive, combinations thereof, and the like. In some embodiments, the pull wire 1114 is not coupled to the tip 1106 and is only coupled to the tube 1118. In some embodiments, the pull wire 1114 is not coupled to the tube 1118 and is only coupled to the tip 1106.

Coupling the pull tube 1114 to the tube 1118 can enhance manufacturing ease and/or device robustness. For example, the pull wire 1114 can be conductive such that the pull wire 1114 is electrically connected to the tube 1118. The pull wire 1114 and/or the tube 1118 can be electrically connected to the tip 11106 (e.g., as shown in FIG. 11E). Detachment of the pull wire 1114 from the tip 1106, for example, would not inhibit the application of energy during a neuromodulation procedure because the tube 1118 can receive the signals from the pull wire 1114 and transmit those signals to the tip 1106.

The steerable stylet 1112 preferably extends out of a lateral side of the needle body 1102. For example, in some embodiments, the stylet 1112 does not exit or extend out of a distal end of the needle body 1102. The steerable stylet 1112 preferably is configured to navigate through soft tissue. For example, in some embodiments, the stylet 1112 cannot penetrate bone or navigate through spongy bone. The steerable stylet 1112 preferably includes an electrode that is used for neuromodulation (e.g., ablation), and thus should not be confused with a guidewire, a guide catheter, etc. In some embodiments, the steerable stylet 1112 is not a hollow steerable component that is then used to guide a separate electrode-bearing tube.

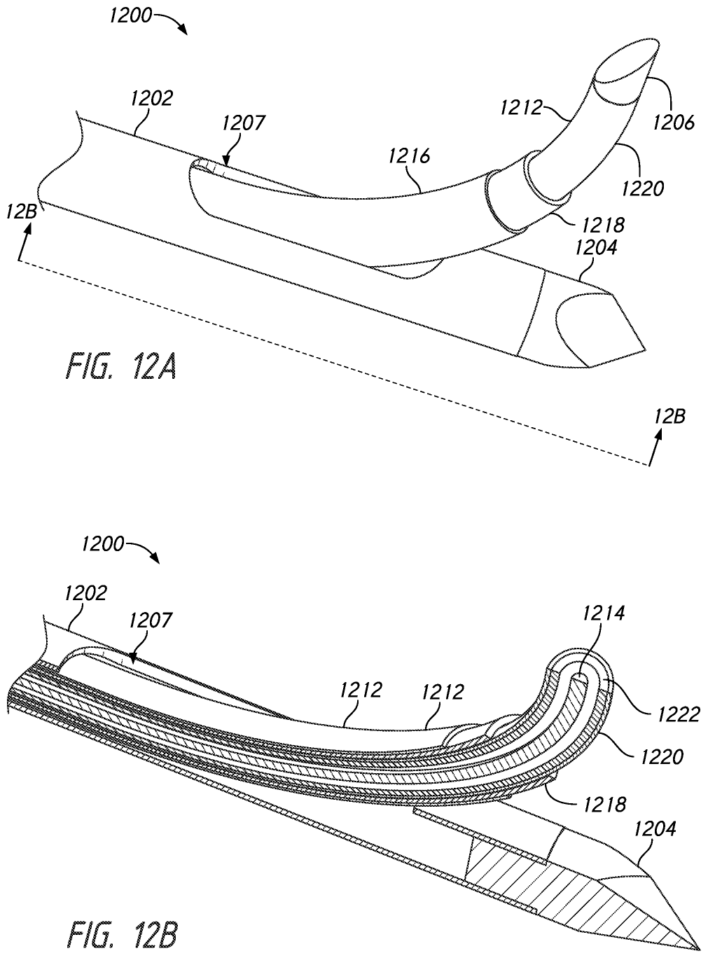

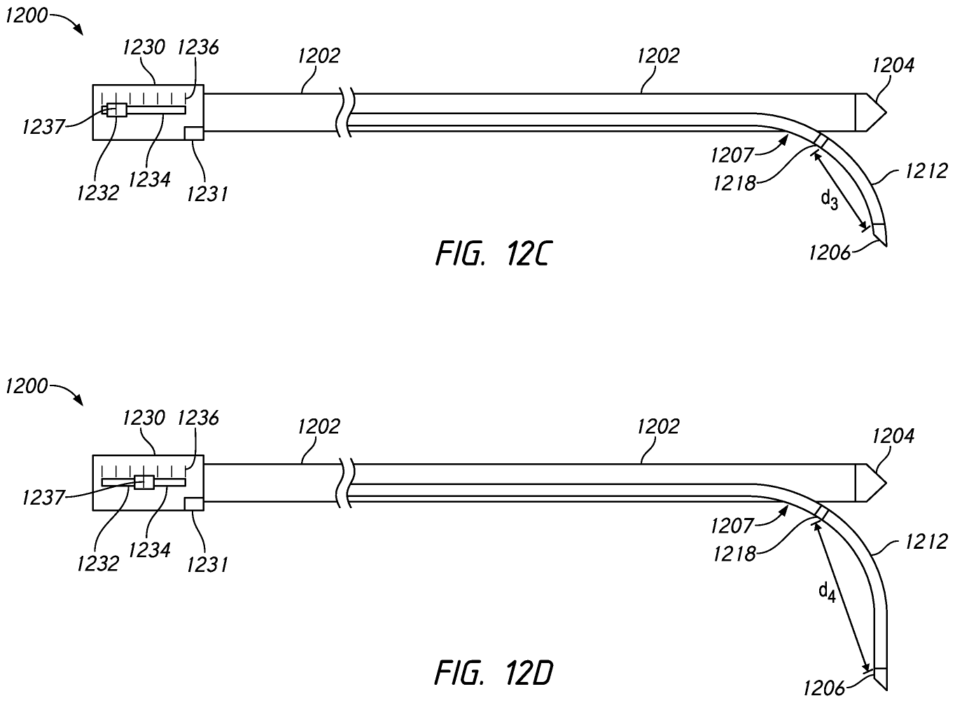

FIG. 12A is a partial perspective view of an example tissue treatment device 1200. The device 1200 may share features with the device 600, the device 900, the device 1000, the device 1100, and/or other tissue treatment devices herein (e.g., radiopaque markers, echogenic surfaces, electrode type, tip type, sensors, materials, dimensions including distances, usage, handle features, connection to a tissue treatment (e.g., nerve modulation (e.g., ablation)) system, movable ramp, etc.). The treatment device 1200 comprises a needle body 1202. The distal end of the needle body 1202 comprises a tissue-penetrating tip 1204. In some embodiments, the tip 1204 may comprises a first electrode. In some embodiments, the tip 1204 and/or the needle body 1202 is free of an electrode. The needle body 1202 comprises an aperture 1207. The needle body 1202 may comprise a longitudinally-extending lumen in communication with the aperture 1207. The needle body 1202 may be hollow at least proximal to the aperture 1207.

The treatment device 1200 comprises a stylet 1212 configured to exit the aperture 1207 and curve radially away from the needle body 1202. The stylet 1212 may comprise a plurality of layers or tubes. For example, the stylet 1212 shown in FIG. 12A includes a first tube 1216, a second tube 1218, and a third tube 1220. The first tube 1216 may comprise an insulating material. The second tube 1218 may comprise a conductive material. The conductive material of the second tube 1218 that is not covered by the first tube 1216 forms a first electrode. The third tube 1220 may comprise an insulating material. The distal end of the stylet 1212 comprises a tissue-penetrating tip 1206. The tip 1206 comprises a second electrode. In some embodiments, the second electrode can be separate from the tip 1206, for example as described with respect to the device 600.

FIG. 12B is a partial cross-sectional side view of the tissue treatment device 1200 of FIG. 12A along the line 12B-12B. FIG. 12B further shows interaction between the tubes 1216, 1218, 1220 and other components. The tip 1206 is removed for clarity. FIG. 12B shows two additional tubes. The stylet 1212 optionally comprises a fourth tube 1222 and a wire 1214. The fourth tube 1222 may, for example, provide column strength during deployment of the stylet 1212 from the needle body 1202. The wire 1214 may comprise a shape set wire (e.g., comprising nitinol) configured to impart curvature to the stylet 1212 when deployed from the needle body 1202. The wire 1214 may be coupled to the tip 1206. The wire 1214 may provide electrical signals to the tip 1206 such that the tip 1206 is the second electrode. The wire 1214 may be electrically insulated from the tube 1218 by the tube 1220 and/or the tube 1222. The tubes 1214, 1216, 1218, 1220, 1222 are generally coaxial about a longitudinal axis.

The tubes 1214, 1220, 1222 telescope relative to the tubes 1216, 1218. When the stylet 1212 is deployed from the needle body 1202, the tubes of the stylet 1212 extend together until the tubes 1216, 1218 reach a stopping point. Upon further deployment, the tubes 1214, 1220, 1222 continue to advance laterally and distally. The tube 1218 comprises a first electrode and the tip 1206 comprises a second electrode such that the distance between the first and second electrodes changes upon additionally deployment of the stylet 1212.

FIG. 12C is a schematic diagram of the tissue treatment device 1200 of FIG. 12A. The device 1200 comprises a handle 1230. The handle 1230 may share features with the handle 630, 930, 1030 (e.g., electronic circuitry 1231, gearing, etc.). The handle 1230 comprises a knob 1232 configured to slide in a channel 1234. Distally advancing the knob 1232 distally advances the stylet 1212. The handle 1230 comprises indicia 1236 to provide a user with information about the extent of extension of the stylet 1212 and/or the distance between the first electrode (e.g., formed from the tube 1218) and the second electrode (e.g., formed from the tip 1206). For example, the indicia 1236 aligned with the knob indicia 1237 in FIG. 12C can correspond to a first distance d₃ between the first electrode and the second electrode.

FIG. 12D is another schematic diagram of the tissue treatment device 1200 of FIG. 12A. FIG. 12D shows the knob 1232 advanced further through the channel 1234. The stylet 1212, in particular the tip 1206, has advanced further out of the needle body 1202, but the position of the tube 1218 is not changed. In some embodiments, the handle 1230 can comprise a detent, indicia, or the like to indicate that the tube 1218 has reached a stopping point. The indicia 1236 aligned with the knob indicia 1237 in FIG. 12D can correspond to a second distance d₄ between the first electrode and the second electrode.

In some embodiments, the first electrode is close enough to the distal end of the device 1200 that an ablation zone between the first electrode and the second electrode can be close to a bone or other surface. The wire 1214 can help the stylet 1212 to take a sharp turn even when advanced out of the needle body 1202 fairly close to the tip 1204. The electrical connections of the device 1200 can all be made at the stylet 1212, which can simplify manufacturing.

The veneered stylet 1212 preferably extends out of one lateral side of the needle body 1202. For example, in some embodiments, the device 1200 does not comprise two stylets exiting or extending out of the needle body 1202. The veneered stylet 1212 preferably includes the first and second electrodes both residing in soft tissue. For example, in some embodiments, the device 1200 does not comprise a first electrode configured to reside in a blood vessel and a second electrode configured to reside in soft tissue.

FIGS. 13A-13D are schematic depictions of example lesions. Different types of electrode devices produce different sizes and shapes of lesions, even when operated under the same or similar radiofrequency signals.

FIG. 13A shows an example lesion 1300 formed using a protruding electrode (PE) probe 1302 including an electrode 1304 deployed from a needle or cannula 1306 (e.g., Venom having a 10 mm active tip from Stryker of Kalamazoo, Michigan). Monopolar energy was applied to both the electrode 1304 and the needle 1306 at 80° C. for 150 seconds. The energy produces a lesion 1300 having a thickness T₁ of 6.9 mm, a length L₁ of 11.7 mm, and a volume of 215 mm³. The electrode 1304 is deployed to make the lesion 1300 larger, and the device does not emit bipolar energy between the probe 1302 and the electrode 1304.

FIG. 13B shows an example lesion 1310 formed using a cooled probe 1312 (e.g., CRK-17-100-4 having a 4 mm active tip from Halyard Health of Alpharetta, Georgia). Monopolar energy was applied at 60° C. for 150 seconds. The energy produces a lesion 1310 having a thickness T₂ of 11.2 mm, a length L₂ of 10.7 mm, and a volume of 595 mm³.

FIG. 13C shows an example lesion 1320 formed using a monopolar probe 1322 (e.g., Venom having a 10 mm active tip without the electrode deployed). Monopolar energy was applied to the probe 1322 at 80° C. for 150 seconds. The energy produces a lesion 1320 having a thickness T₃ of 5.9 mm, a length L₃ of 10.8 mm, and a volume of 169 mm³.

FIG. 13D shows an example lesion 1330 formed using a multi-tine (MT) probe 1332 including two electrodes 1334 deployed from a needle 1336 (e.g., Nimbus from Stratus Medical of Magnolia, Texas). Monopolar energy was applied to the probe 1332 at 75° C. for 80 seconds. The energy produces a lesion 1330 having a thickness T₄ of 9 mm, a length L₄ of 10 mm, and a volume of 467 mm³. The electrodes 1334 are deployed to make the lesion 1330 larger, and the device does not emit bipolar energy between the probe 1332 and the electrodes 1334.

FIG. 13E is another schematic depiction of an example lesion 1350 using an ablation device 1352 having features described herein (e.g., the device 600, 800, 900, 1000, 1100, 1200). Bipolar energy was applied between a first electrode 1354 and a second electrode 1356 on a deployable stylet 1358. The energy produces a lesion 1350 having a thickness T₅ of 20 mm, a length L₅ of 5 mm, and a volume of 1,047 mm³. The lesion 1350 has a thickness T₅ that is significantly greater than (e.g., more than double) the thicknesses T₁, T₂, T₃, T₄. The thickness T₅ may be between about 1 mm and about 100 mm (e.g., about 1 mm, about 5 mm, about 10 mm, about 15 mm, about 20 mm, about 25 mm, about 30 mm, about 40 mm, about 50 mm, about 75 mm, about 100 mm, ranges between such values, and the like) depending on the subject being treated. For example, a mouse may have a very small hip joint capsule while an elephant may have a very large hip joint capsule. The lesion 1350 has a length L5 that is significantly smaller than (e.g., less than half of) the lengths L₁, L₂, L₃, L₄. The length L₅ may be substantially constant over a range of thickness T₅. The lesion 1350 has a thickness T₅ to length L₅ (T₅:L₅) ratio of 20:5 (4:1), which is significantly greater than (e.g., more the four times) the ratios T₁:L₁ (6.9/11.7 (0.57:1)), T₂:L₂ (11.2/10.7 (1.05:1)), T₃:L₃ (5.9/10.8 (0.55:1)), T₄:L₄ (9/10 (0.9:1)). In some embodiments, a lesion produced by the devices described herein have a thickness to length ratio greater than about 1.25:1 and/or between about 1.25:1 and about 10:1 (e.g., about 1.25:1, about 1.5:1, about 2:1, about 2.5:1, about 3:1, about 3.5:1, about 4:1, about 4.5:1, about 5:1, about 6:1, about 8:1, about 10:1, ranges between such values, and the like). Greater thicknesses and/or ratios can be achieved, for example, by further extending an electrode on a stylet further from an electrode on a needle from which the stylet extends and/or further from an electrode on the stylet proximate to a needle from which the stylet extends. The lesion 1350 can have a thin long shape that is better suited for denervation and/or is more adjustable for the specific application than the shapes of the lesions 1300, 1310, 1320, 1330.

FIG. 14 is a schematic front (left) and side (right) depiction of example lesion 1400 using an ablation device 1402 having features described herein (e.g., the device 600, 800, 900, 1000, 1100, 1200). The area bounded by the solid line is the actual lesion 1400 and the area bounded by the dashed line is the expected lesion 1401. The expected lesion 1400 and the actual lesion 1401 are very close to each other. The device 1402 is designed specifically for the anatomy and tissue composition. For example, FIG. 14 also shows layers including bone 1410 (0.03 Siemens/meter (S/m) conductivity), adipose tissue 1412 (0.1 S/m), and muscle 1414 (0.54 S/m), and schematically shows the connective tissue layers 1416 protecting the nerve including the epineurium (0.08 S/m), endoneurium (0.08 S/m), and perineurium (0.002 S/m). The device 1402 focuses the electrical current in the target area and across the nerve, reduces (e.g., minimizes) current loss and/or damage to muscle 1414, and/or creates a more reproducible ablation zone 1401.

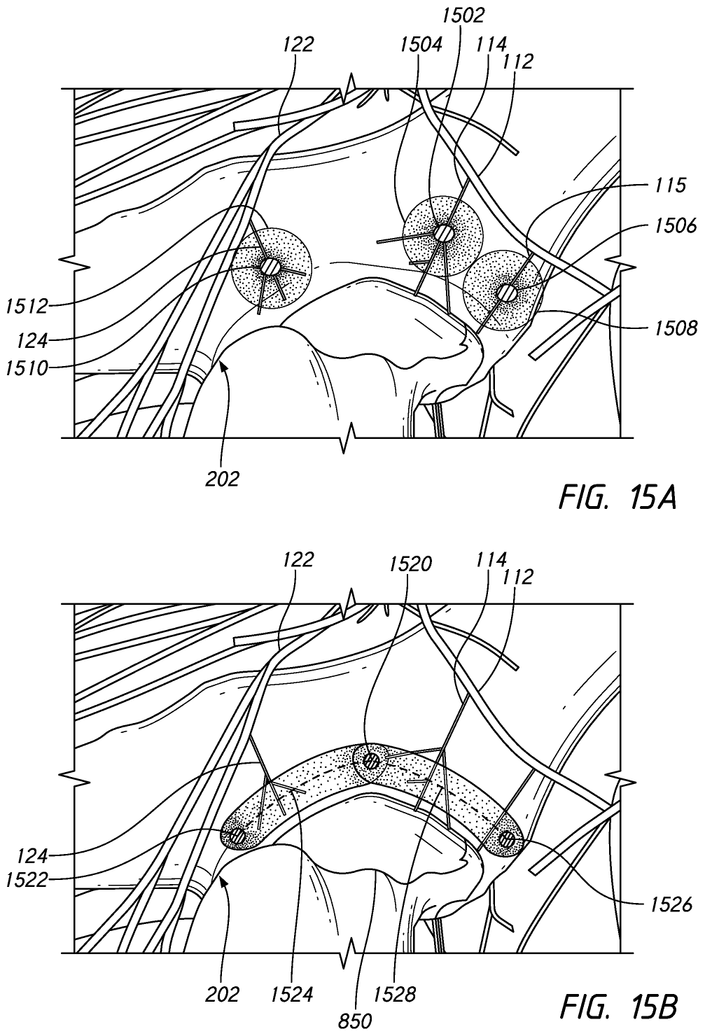

FIG. 15A is an example method of treating hip joint pain. For example, the method illustrated in FIG. 15A may be performed using one or more of the probes 1302, 1312, 1322, 1332 to form one or more of the lesions 1300, 1310, 1320, 1330. The method comprises modulating (e.g., denervating, ablating, etc.) an articular branch 124 from the sciatic nerve 122 and an articular branch 114 from the cranial gluteal nerve 112. The method optionally comprises modulating an articular branch 104 from femoral nerve 102 (e.g., FIGS. 1A, 1B, 8A). The method optionally comprises modulating an articular branch 134 from the obturator nerve 132 (e.g., FIGS. 1B, 5, 8B).

A user can insert a treatment device at a first point 1502. The first point 1502 may attempt to target the articular branch 114. Facets of the hip joint capsule 202 may be used to help determine a position of the first point 1502, although it may be difficult to use landmarks for positioning. Because the T:L ratio of the lesion will be less than 1.25, the orientation of the device is not important. Imaging (e.g., fluoroscopy, ultrasound, etc.) may be used to verify the position of the treatment device prior to modulation. A lesion 1504 may then be created by applying energy to the treatment device. The lesion 1504 has a T:L ratio less than 1.25 and extends into the muscle. If the first point 1502 is not well targeted, the articular branch 114 may be entirely missed, as the low T:L ratio generally cannot account for nerve location variability. The user can insert the treatment device at a second point 1506 to attempt to modulate a second articular branch 115. A lesion 1508 may then be created by applying energy to the treatment device. The lesion 1508 has a T:L ratio less than 1.25 and extends into the muscle. If the second point 1504 is not well targeted, the articular branch 115 may be entirely missed. The user can insert the treatment device at a third point 1510 to attempt to modulate the articular branch 124. A lesion 1512 may then be created by applying energy to the treatment device. The lesion 1512 has a T:L ratio less than 1.25 and extends into the muscle. If the third point 1510 is not well targeted, the articular branch 124 may be entirely missed. The user therefore may have performed at least three separate insertions and at least three ablations and missed the articular branches 114, 115, 124 altogether, but affected muscle tissue around at least three points of the hip joint capsule 202. Ablating muscle tissue can cause additional pain. The lesions 1504, 1508, 1512 may also or alternatively ablate critical structures.

FIG. 15B is another example method of treating hip joint pain. The method of FIG. 15B may share features with the method of FIGS. 8A and 8B. The method comprises modulating (e.g., denervating, ablating, etc.) an articular branch 124 from the sciatic nerve 122 and an articular branch 114 from the cranial gluteal nerve 112. The method optionally comprises modulating an articular branch 104 from femoral nerve 102 (e.g., FIGS. 1A, 1B, 8A). The method optionally comprises modulating an articular branch 134 from the obturator nerve 132 (e.g., FIGS. 1B, 5, 8B).

Using a dorsal and caudal approach, a user can insert a treatment device as described herein (e.g., the device 600, 800, 900, 1000, 1100, 1200) at a first point 1520. The first point 1520 may be an apex of a hip joint capsule. The user can advance the treatment device until the distal end contacts bone. Other approaches are also possible. Facets of the hip joint capsule 202 may be used to help determine a position of the first point 1520. Imaging can help to orient the treatment device so that the stylet is deployed until the second point 1522. The acetabulum/acetabular rim (socket) and femoral head 850, for example, may be used to help determine a position of the second point 1522. The acetabulum is a concave/curved surface. The devices described herein may be uniquely designed to allow electrode positioning on bone around a concave/curved structure. The deployed stylet may extend distal to the tip of the needle body, for example by curving downward along a bone. Imaging (e.g., fluoroscopy, ultrasound, etc.) may be used to verify the position of the treatment device prior to modulation. Terms such as “first,” second, “third,” etc. provide nomenclature that can help distinguish between different points or other multiple items discussed herein, and do not necessarily describe an order, a preference, a hierarchy, etc. For example, in some embodiments, the treatment device may be inserted at the second point 1522 and extended to the first point 1520. For example, the second point 1522 may be easier to identify than the first point 1520 when percutaneously positioning the treatment device. The acetabulum is bounded dorsally, cranially, and caudally by the acetabular rim. The targeted modulations may occur just above this rim when targeting the articular branch 124 of the sciatic nerve 122 and/or the articular branch 114 of the cranial gluteal nerve 112. Using borders (e.g., dorsal, cranial, caudal) of the hip joint or acetabulum can help to identify the rim, which may be termed the dorsal rim and/or the ventral rim). Although certain examples of insertion and extension points are provided herein, other insertion and extension points are also possible, for example others that would create a path that would include an articular branch nerve of interest. Bipolar radiofrequency energy can be applied between the first electrode and the second electrode. The energy will extend along the path 1524, forming a substantially linear or curved/arcuate ablation zone (e.g., having a T:L ratio greater than about 1.25, as described with respect to FIGS. 13E and 14; measuring the curved thickness of the outside of the ablation zone in the case of curved/arcuate). The curvature of the ablation zone may follow the curvature of the bone, and bone is reflective of RF energy. The articular branch 124 from the sciatic nerve 122 lies in the path 1524 such that the RF energy can modulate the articular branch 124. The stylet can be retracted back into the needle body. After modulating the articular branch nerve 124, the pain is reduced. Pain reduction can be measured, for example, by walking evaluation, a biped station, rotation with external abduction, subluxation and iliopsoas, combinations thereof, a distraction index, etc. The test(s) can be performed before a procedure to establish a preoperative baseline, and then at one or more intervals after the procedure (e.g., one day, two days, one week, two weeks, one month, three months, six months, etc.). Imaging can also or alternatively be used to evaluate the tissue for signs of recovery.

In some embodiments, the user can rotate the treatment device (e.g., between about 100° and about 160°) to orient the treatment device so that the stylet is deployed until the third point 1526. Facets of the hip joint capsule 202 may be used to help determine a position of the third point 1526. Imaging (e.g., fluoroscopy, ultrasound, etc.) may be used to verify the position of the treatment device prior to modulation. Bipolar radiofrequency energy can be applied between the first electrode and the second electrode. The energy will extend along the path 1528, forming a substantially linear or curved/arcuate ablation zone (e.g., having a T:L ratio greater than about 1.25, as described with respect to FIGS. 13E and 14; measuring the curved thickness of the outside of the ablation zone in the case of curved/arcuate). The curvature of the ablation zone may follow the curvature of the bone, and bone is reflective of RF energy The articular branch 114 from the cranial gluteal nerve 112 lies in the path 1528 such that the RF energy can modulate the articular branch 114. After modulating the articular branch nerve 114, the pain is reduced. The stylet can be retracted back into the needle body. Rotating the treatment device between energy application can reduce the number of puncture sites (e.g., only a single puncture site to denervate the entire hip joint capsule 202), which can reduce soft tissue trauma and a number of possible infection sites. A smaller amount of rotation is also possible, for example to make sure that the first tissue was treated enough to capture the articular branch nerve. The paths 1524, 1528 extending along the hip joint capsule 202 can account for any nerve location variation that could occur. If the capsule 202 as illustrated in FIG. 15B is considered a clock face, the lesions may be created between the 12:00 position and the 3:00 position and between the 12:00 position and the 9:00 position, for example. The method can ablate two segments or paths to ablate two quadrants of a capsule from a single percutaneous insertion position. The lesions created along the paths 1524, 1528 can direct ablation away from critical structures and muscle. The method can simplify positioning using visible anatomical landmarks. The ablation zones have an adjustable length to account for various sizes of capsules 202 from subject to subject and across various species of subjects.

Although described herein with respect to hip joint pain in canines, the methods and systems described herein may be suitable for other joints and species. For example, hip pain in canines, equines, felines (including big cats such as lions and tigers), bovines, porcines, pachyderms, and other species maybe treated by modulating an articular branch nerve. Some bulls are not able to naturally mate due to hip joint pain inhibiting the bull from being able to mount the heifer, usually leaving artificial insemination and pharmaceuticals as the only options. Modulation as described herein can relieve the pain to allow natural insemination, and can avoid pharmaceuticals that might otherwise wind up in beef from the bull. For additional examples, stifle, elbow, and/or spine pain in canines, equines, felines, bovines, porcines, pachyderms, and other species maybe treated by modulating an articular branch nerve.

The foregoing description and examples has been set forth merely to illustrate the disclosure and are not intended as being limiting. Each of the disclosed aspects and examples of the present disclosure may be considered individually or in combination with other aspects, examples, and variations of the disclosure. In addition, unless otherwise specified, none of the steps of the methods of the present disclosure are confined to any particular order of performance. Modifications of the disclosed examples incorporating the spirit and substance of the disclosure may occur to persons skilled in the art and such modifications are within the scope of the present disclosure. Furthermore, all references cited herein are incorporated by reference in their entirety.

The features of the devices 600, 900, 1000, 1100, 1200 can be combined in some embodiments. Non-limiting examples include: a device comprising a movable ramp, a steerable stylet, and a veneered stylet; a device comprising a movable ramp and a steerable stylet; a device comprising a movable ramp and a veneered stylet; and a device comprising a steerable and veneered stylet, etc.

While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various examples described and the appended claims. Further, the disclosure herein of any particular feature, aspect, method, property, characteristic, quality, attribute, element, or the like in connection with an example can be used in all other examples set forth herein. Any methods disclosed herein need not be performed in the order recited. Depending on the example, one or more acts, events, or functions of any of the algorithms, methods, or processes described herein can be performed in a different sequence, can be added, merged, or left out altogether (e.g., not all described acts or events are necessary for the practice of the algorithm). In some examples, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each example. Additionally, all possible combinations, subcombinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some examples may be performed using the sequence of operations described herein, while other examples may be performed following a different sequence of operations.

Conditional language used herein, such as, among others, “can,” “might,” “may,” “e.g.,” and the like, unless specifically stated otherwise, or otherwise understood within the context as used, is generally intended to convey that some examples include, while other examples do not include, certain features, elements, and/or states. Thus, such conditional language is not generally intended to imply that features, elements, blocks, and/or states are in any way required for one or more examples or that one or more examples necessarily include logic for deciding, with or without author input or prompting, whether these features, elements and/or states are included or are to be performed in any particular example.

The methods disclosed herein may include certain actions taken by a practitioner; however, the methods can also include any third-party instruction of those actions, either expressly or by implication. For example, actions such as “positioning a catheter” include “instructing positioning of a catheter.”

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, 15%, etc.). For example, “about 4 mm” includes “4 mm.” Phrases preceded by a term such as “substantially” include the recited phrase and should be interpreted based on the circumstances (e.g., as much as reasonably possible under the circumstances). For example, “substantially linear” includes “linear.” Unless stated otherwise, all measurements are at standard conditions including temperature and pressure.

The phrase “at least one of” is intended to require at least one item from the subsequent listing, not one type of each item from each item in the subsequent listing. For example, “at least one of A, B, and C” can include A; B; C; A and B; A and C; B and C; or A, B, and C.

What is claimed is:

1. A minimally invasive method of modulating an articular nerve branch to treat hip joint pain in a subject, the method comprising:
imaging the hip joint using at least one of ultrasound or fluoroscopy to identify anatomical landmarks;
percutaneously inserting a treatment device to a first point determined by the anatomical landmarks until a distal end of the treatment device contacts bone;
extending a stylet out of a needle body of the treatment device, wherein extending the stylet comprises sliding the stylet along the bone to a second point, the needle body comprising a first electrode and the stylet comprising a second electrode;
imaging using at least one of ultrasound or fluoroscopy to confirm a first location of the first electrode and the second electrode relative to the anatomical landmarks;
applying bipolar radiofrequency energy to the first electrode and the second electrode to ablate first tissue between the first point and the second point, the first tissue including an articular branch nerve of a sciatic nerve;
retracting the stylet into the needle body;
rotating the treatment device at the first point;
extending the stylet out of the needle body of the treatment device, wherein extending the stylet comprises sliding the stylet along the bone to a third point;
imaging using at least one of ultrasound or fluoroscopy to confirm a second location of the first electrode and the second electrode relative to the anatomical landmarks;
applying bipolar radiofrequency energy to the first electrode and the second electrode to ablate second tissue between the first point and the third point, the second tissue including an articular branch nerve of a cranial gluteal nerve;
retracting the stylet into the needle body; and
retracting the treatment device out of the subject.

2. A minimally invasive method of modulating an articular nerve branch to treat hip joint pain in a subject, the method comprising:
imaging the hip joint using at least one of ultrasound or fluoroscopy to identify anatomical landmarks;
percutaneously inserting a treatment device to a first point determined by the anatomical landmarks until a distal end of the treatment device contacts bone;
extending a stylet out of a needle body of the treatment device, wherein extending the stylet comprises sliding the stylet along the bone to a second point, the needle body comprising a first electrode and the stylet comprising a second electrode;
imaging using at least one of ultrasound or fluoroscopy to confirm a first location of the first electrode and the second electrode relative to the anatomical landmarks;
applying bipolar radiofrequency energy to the first electrode and the second electrode to ablate first tissue between the first point and the second point, the first tissue including an articular branch nerve of a sciatic nerve;
retracting the stylet into the needle body;
retracting the treatment device out of the subject;
percutaneously inserting the treatment device to a third point determined by the anatomical landmarks until the distal end of the treatment device contacts the bone;
extending the stylet out of the needle body of the treatment device, wherein extending the stylet comprises sliding the stylet along the bone to a fourth point;
imaging using at least one of ultrasound or fluoroscopy to confirm a second location of the first electrode and the second electrode relative to the anatomical landmarks;
applying bipolar radiofrequency energy to the first electrode and the second electrode to ablate second tissue between the third point and the fourth point, the second tissue including an articular branch nerve of a cranial gluteal nerve;
retracting the stylet into the needle body; and
retracting the treatment device out of the subject.

3. The method of claim 1 or 2, further comprising:
percutaneously inserting the treatment device to a fifth point determined by the anatomical landmarks until a distal end of the treatment device contacts bone;
extending the stylet out of the needle body of the treatment device, wherein extending the stylet comprises sliding the stylet along the bone to a sixth point;
imaging using at least one of ultrasound or fluoroscopy to confirm a third location of the first electrode and the second electrode relative to the anatomical landmarks;
applying bipolar radiofrequency energy to the first electrode and the second electrode to ablate third tissue between the fifth point and the sixth point, the third tissue including an articular branch nerve of a femoral nerve;
retracting the stylet into the needle body; and
retracting the treatment device out of the subject.

4. The method of claim 1 or 2, further comprising:
percutaneously inserting the treatment device to a seventh point determined by the anatomical landmarks until a distal end of the treatment device contacts bone;
extending the stylet out of the needle body of the treatment device, wherein extending the stylet comprises sliding the stylet along the bone to an eighth point;
imaging using at least one of ultrasound or fluoroscopy to confirm a fourth location of the first electrode and the second electrode relative to the anatomical landmarks;
applying bipolar radiofrequency energy to the first electrode and the second electrode to ablate fourth tissue between the seventh point and the eighth point, the fourth tissue including an articular branch nerve of an obturator nerve;
retracting the stylet into the needle body; and
retracting the treatment device out of the subject.

5. The method of claim 1 or 2, wherein the subject is a quadruped.

6. A method of modulating an articular nerve branch to treat hip joint pain in a subject, the method comprising:
percutaneously inserting a treatment device to a first point until a distal end of the treatment device contacts bone;
extending a stylet out of a needle body of the treatment device, wherein extending the stylet comprises sliding the stylet along the bone to a second point, the needle body comprising a first electrode and the stylet comprising a second electrode;
applying bipolar radiofrequency energy to the first electrode and the second electrode to ablate first tissue between the first point and the second point, the first tissue including an articular branch nerve of a first one of a sciatic nerve, a cranial gluteal nerve, a femoral nerve, or an obturator nerve;
retracting the stylet into the needle body.

7. The method of claim 6, further comprising repeating the percutaneously inserting, extending, and applying to ablate second tissue including an articular branch nerve of a second one of the sciatic nerve, the cranial gluteal nerve, the femoral nerve, or the obturator nerve.

8. The method of claim 7, wherein the first one is the articular branch of the sciatic nerve and the second one is the articular branch of the cranial gluteal nerve, or wherein the first one is the articular branch of the cranial gluteal nerve and the second one is the articular branch of the sciatic nerve.

9. The method of claim 7, further comprising repeating the percutaneously inserting, extending, and applying to ablate second tissue including an articular branch nerve of a third one of the sciatic nerve, the cranial gluteal nerve, the femoral nerve, or the obturator nerve.

10. The method of claim 9, further comprising repeating the percutaneously inserting, extending, and applying to ablate second tissue including an articular branch nerve of a fourth one of the sciatic nerve, the cranial gluteal nerve, the femoral nerve, or the obturator nerve.

11. The method of any one of claims 6 to 10, wherein the subject is a quadruped.

12. A minimally invasive method of modulating an articular nerve branch to treat pain in a joint of a subject, the method comprising:
identifying an articular branch nerve emanating from a nerve innervating the joint; and
percutaneously modulating the articular branch nerve, wherein after modulating the articular branch nerve the pain is reduced.

13. The method of claim 12, wherein the joint comprises a hip.

14. The method of claim 13, wherein identifying the articular branch nerve comprises:
acquiring a hip joint reference point corresponding to a radiographically identifiable anatomical feature of the hip joint; and
generating coordinates for a target treatment site as a function of a calculated distance from the reference point.

15. The method of any one of claims 12 to 14, wherein the nerve comprises at least one of the cranial gluteal nerve, femoral nerve, sciatic nerve, or obturator nerve.

16. The method of any one of claims 12 to 14, wherein modulating the articular branch nerve comprises modulating the articular branch nerve at a target site.

17. The method of claim 16, wherein the target site is external to the joint.

18. The method of claim 16, wherein the target site comprises a portion of the articular branch nerve proximate a junction between the articular branch nerve and the nerve.

19. The method of claim 16, wherein the target site comprises a portion of the articular branch nerve proximate a terminus of the articular branch nerve.

20. The method of claim 16, wherein the target site comprises a portion of the articular branch nerve between a junction between the articular branch nerve and the nerve and a terminus of the articular branch nerve.

21. The method of any one of claims 12 to 14, wherein modulating the articular branch nerve comprises:
percutaneously inserting a treatment device to a first point;
extending a stylet out of a needle body of the treatment device, the needle body comprising a first electrode and the stylet comprising a second electrode;
applying energy to the treatment device to ablate tissue including the articular branch nerve.

22. The method of claim 21, wherein applying the energy comprises applying radiofrequency energy.

23. The method of claim 22, wherein the radiofrequency energy is bipolar.

24. The method of any one of claims 12 to 14, further comprising modulating the nerve.

25. The method of any one of claims 12 to 14, wherein the subject is a quadruped.

26. The method of claim 25, wherein the subject is a canine.

27. The method of any one of claims 12 to 14, wherein the subject has osteoarthritis or degenerative joint disease.

28. A device for treating tissue, the device comprising:
a needle body comprising:
a tissue penetrating tip; and
an aperture in a side of the needle body;
a first electrode;
a stylet configured to radially extend from the aperture of the needle body, the stylet comprising:
a second electrode; and
a tissue penetrating tip;
a handle configured to be coupled to a tissue treatment system, the handle comprising a stylet mechanism configured to longitudinally move the stylet relative to the needle body.

29. The device of claim 28, wherein the needle body comprises a first temperature sensor.

30. The device of claim 29, wherein the stylet comprises a second temperature sensor.

31. The device of claim 28, wherein the stylet is configured to radially extend from the aperture of the needle body along a curved path.

32. The device of claim 28, wherein the stylet mechanism comprises a knob configured to slide along a path.

33. The device of claim 28, wherein the stylet mechanism comprises the handle being rotatable relative to the needle body.

34. The device of claim 28, wherein the stylet mechanism comprises a rotatable knob.

35. The device of claim 28, wherein the needle body comprises the first electrode.

36. The device of claim 28, wherein the tissue penetrating tip of the needle body comprises the first electrode.

37. The device of claim 28, wherein the tissue penetrating tip of the stylet comprises the first electrode.

38. The device of claim 28, wherein the tissue penetrating tip of the stylet comprises the first electrode.

39. The device of claim 28, wherein the stylet mechanism comprises indicia configured to inform a user about an extend of longitudinal movement of the stylet.

40. The device of claim 28, wherein the stylet mechanism comprises detents configured to inform a user about an extend of longitudinal movement of the stylet.

41. The device of claim 28, further comprising electronic circuitry configured to track use information.

42. The device of any one of claims 28 to 41, further comprising a longitudinally movable ramp, wherein a longitudinal position of the ramp affects a longitudinal position at which the stylet radially extends from the aperture of the needle body.

43. The device of claim 42, wherein the handle comprises a ramp mechanism configured to longitudinally move the ramp relative to the needle body.

44. The device of any one of claims 28 to 41, wherein the stylet is steerable.

45. The device of claim 44, wherein the stylet is steerable in one direction.

46. The device of claim 44, wherein the stylet is steerable in two directions.

47. The device of claim 46, wherein the two directions are on one plane.

48. The device of claim 44, wherein the stylet comprises one steering wire.

49. The device of claim 44, wherein the stylet comprises two steering wires.

50. The device of claim 44, wherein the stylet comprises a tube.

51. The device of claim 50, wherein the tube comprises a first plurality of kerfs on a first side of the tube and a second plurality of kerfs on a second side of the tube opposite the first side.

52. The device of claim 51, wherein at least one of a shape, size, or spacing of the first plurality of kerfs is different than at least one of a shape, size, or spacing of the second plurality of kerfs.

53. The device of claim 50, wherein a distal portion of the tube comprises a pattern and material bent radially inwardly to attach a steering wire between the material and an inner surface of the tube.

54. The device of claim 44, wherein the stylet mechanism comprises a knob configured to slide along a path and to rotate relative to the needle body, wherein rotation of the knob is configured to steer the stylet.

55. The device of any one of claims 28 to 41, wherein the stylet comprises the first electrode and the second electrode longitudinally movable relative to each other.

56. The device of claim 55, wherein the stylet comprises a first tube comprising the first electrode and a second tube comprising a second electrode, the second tube in telescoping arrangement with the first tube.

57. The device of claim 55, wherein the stylet comprises a tube comprising a shape memory material.

58. A minimally invasive method of treating pain in an arcuate joint capsule of a subject, the method comprising:
percutaneously positioning a treatment device at a first point along the capsule;
extending a stylet to a second point along the capsule;
applying energy to the device to create a first lesion along the capsule;
retracting the stylet;
extending the stylet to a third point along the capsule; and
applying energy to the device to create a second lesion along the capsule, the second lesion at an angle to the first lesion.

59. The method of claim 58, wherein the first lesion has a thickness to length ratio between 1.25:1 and 10:1.

60. The method of claim 58, wherein the second lesion has a thickness to length ratio between 1.5:1 and 10:1.

61. The method of any one of claims 58 to 60, wherein the capsule is a hip joint capsule.

62. The method of any one of claims 58 to 60, wherein the subject is a quadruped.

63. A minimally invasive method of treating pain in a subject, the method comprising:
positioning a treatment device at a first point;
extending a stylet from the treatment device to a second point, wherein after extending the stylet to the second point a first electrode is proximate to or touching a bone of the subject and the stylet comprises a second electrode proximate to or touching the bone; and
applying energy to the device to create a lesion along the bone, the lesion following the curvature of the bone.

64. The method of claim 63, wherein applying the energy comprises applying bipolar radiofrequency energy.

65. The method of claim 63, wherein the bone includes an arcuate rim.

66. The method of claim 63, wherein the treatment device comprises the first electrode.

67. The method of claim 63, wherein the stylet comprises the first electrode.

68. The method of any one of claims 63 to 67, wherein the bone is part of a hip joint capsule.

69. The method of any one of claims 63 to 67, wherein the subject is a quadruped.

70. A treatment system having one or more of the features described in the foregoing description.

71. A tissue treatment system having one or more of the features described in the foregoing description.

72. A method of modulating an articulator nerve having one or more of the features described in the foregoing description.
